SEQUENCE LISTING (1 sequence)

SEQ ID NO: 1 - DNA, 12 bp, synthetic construct
  source 1..12: mol_type = other DNA
tatggtcgtcga

(19) United States
(12) Patent Application Publication
(10) Pub. No.: US 2024/0249797 A1
(43) Pub. Date: Jul. 25, 2024

(54) ADAPTIVE BASE CALLING SYSTEMS AND METHODS

(71) Applicant: Ultima Genomics, Inc., Fremont, CA (US)

(72) Inventors: Yoav ETZIONI, Tel Aviv (IL); Edward PERELMAN, Lehavim (IL); Asaf HALLE, Petah Tikva (IL)

(21) Appl. No.: 18/424,587

(22) Filed: Jan. 26, 2024

Related U.S. Application Data
(63) Continuation of application No. PCT/US2022/074246, filed on Jul. 28, 2022.
(60) Provisional application No. 63/203,746, filed on Jul. 29, 2021.

(51) Int. Cl. G16B 30/00; C12Q 1/6874; G16B 40/20

(52) U.S. Cl. CPC G16B 30/00; C12Q 1/6874; G16B 40/20

(57) ABSTRACT

Methods for updating a system comprising a sequencer are described herein. In some exemplary methods, the system is updated through generating sequencing data for a plurality of nucleic acid molecule colonies, selecting sequencing data for a subset of the nucleic acid molecule colonies, calling preliminary sequences for the subset of the nucleic acid colonies, mapping the called preliminary sequences to a known reference sequence, and updating the pre-trained sequencer-specific machine-learning model. Also described herein are systems for carrying out such methods and computer readable memory for storing such methods.

### CROSS-REFERENCE TO RELATED APPLICATION

This application claims the priority benefit of U.S. Provisional Patent Application Ser. No. 63/203,746, filed Jul. 29, 2021; the contents of which are incorporated herein by reference in its entirety.

### SUBMISSION OF SEQUENCE LISTING ON ASCII TEXT FILE

The contents of the electronic sequence listing (165272001140SEQLIST.xml; Size: 1,891 bytes; and Date of Creation: Jul. 27, 2022) is herein incorporated by reference in its entirety.

### FIELD OF THE INVENTION

Described herein are methods of updating a system that includes a sequencer for sequencing nucleic acid molecules.

### BACKGROUND

Many nucleic acid sequencers operate by detecting a signal, such as a fluorescence signal, from labeled nucleotides integrated into an extending sequencing primer, which provides information about the sequence of the complementary template strand. The signals are detected and processed to determine the sequence of the template strand. Certain sequencing methods, such as the flow sequencing methods described in U.S. Pat. No. 8,772,473, rely on the association between a detected signal intensity and homopolymer length at a given sequencing flow position. Thus, accurate template strand sequencing relies on an accurate association between signal intensity and homopolymer length.

Sequencers are sensitive devices, and it is important that the detected signal is accurate to correctly identify the sequence of the target nucleic acid molecules. Sequencers are susceptible to instrument drift over time, which can affect the overall accuracy of the sequencing readout.

### BRIEF SUMMARY OF THE INVENTION

Described herein are methods of updating a system comprising a sequencer. Also described herein are systems for carrying out such methods. Further described are computer-readable memory for storing such methods.

In some aspects, provided herein is a method of updating a system comprising a sequencer, the method comprising: receiving, at one or more processors, sequencing data for a plurality of nucleic acid molecule colonies comprising nucleic acid molecules derived from a selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; selecting, using the one or more processors, sequencing data for a subset of the nucleic acid molecule colonies; calling, using the one or more processors, preliminary sequences for the subset of the nucleic acid molecule colonies, comprising inputting the selected sequencing data into a pre trained sequencer-specific machine learning model configured to call a homopolymer length or a homopolymer length likelihood for each sequencing flow step based on the signal intensity values, wherein the pre-trained sequencer specific machine-learning model was previously trained based on sequencing data previously generated using the same sequencer and nucleic acid molecules from the same selected species; mapping, using the one or more processors, the called preliminary sequences to a known reference sequence to identify corresponding reference sequence fragments for the called preliminary sequences; and updating, using the one or more processors, the pre-trained sequencer-specific machine-learning model based on a training data set comprising the selected sequencing data and the corresponding reference sequence fragments.

In some embodiments, the method comprises generating, using the sequencer, the sequencing data. In some embodiments, wherein the pre-trained sequencer-specific machine-learning model was previously updated based on penultimate sequencing data generated using the same sequencer and penultimate nucleic acid molecules from the same selected species.

In some embodiments, the pre-trained sequencer-specific machine-learning model was previously updated by a method comprising: generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step; selecting penultimate sequencing data for a subset of the penultimate nucleic acid molecule colonies; calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species; mapping the called penultimate preliminary sequences to the known reference sequence to identify corresponding penultimate reference sequence fragments for the called penultimate preliminary sequences; and updating the penultimate pre-trained sequencer-specific machine-learning model based on a penultimate training data set comprising the selected penultimate sequencing data and the corresponding penultimate reference sequence fragments.

In some embodiments, the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

In some embodiments, the pre-trained sequencer-specific machine-learning model was previously initialized using sequencing data previously generated using the same sequencer and nucleic acid molecules from a different selected species.

In some embodiments, the different selected species has a smaller genome than the selected species. In some embodiments, the different selected species is a bacterial species or a viral species. In some embodiments, the different selected species is Escherichia coli.

In some embodiments, the selected species is a primate. In some embodiments, the selected species is a human.

In some embodiments, the sequencer-specific machine-learning model is a neural network. In some embodiments, the sequencer-specific machine-learning model is a convoluted neural network.

In some embodiments, the sequencing data comprises, for each nucleic acid molecule colony, a vector comprising a signal intensity value at each sequencing flow step.

In some embodiments, updating the pre-trained sequencer-specific machine-learning model based on the training data set comprises iteratively updating the pre-trained sequencer-specific machine-learning model using the same training data set until a predetermined quality control threshold is met or surpassed. In some embodiments, the predetermined quality control threshold is a convergence threshold.

In some embodiments, updating the pre-trained sequencer-specific machine-learning model based on the training data set comprises iteratively updating the pre-trained sequencer-specific machine-learning model using the same training data set until a change in a quality control value between iterations is below a predetermined threshold. In some embodiments, the predetermined threshold is a convergence threshold.

In some embodiments, the selected sequencing data for the subset of the nucleic acid molecule colonies is randomly selected.

Also provided herein is a method of determining a sequence of a target nucleic acid molecule, comprising: updating a system according to the method of any one of the above embodiments, wherein the plurality of nucleic acid molecule colonies comprises a colony comprising the target nucleic acid molecule; inputting, using the one or more processors, the sequencing data for the colony comprising the target nucleic acid molecule into the updated sequencer-specific machine-learning model; and calling, using the one or more processors, for the target nucleic acid molecule, a homopolymer length for each sequencing flow step using the updated sequencer-specific machine-learning model.

Also provided herein is a system, comprising a sequencer; one or more processors; a computer-readable memory; a pre-trained sequencer-specific machine-learning model stored in the computer-readable memory, wherein the pre-trained sequencer-specific machine-learning model is configured to call a homopolymer length or a homopolymer length likelihood for each sequencing flow step based on signal intensity values, wherein the pre-trained sequencer-specific machine-learning model was previously trained based on sequencing data previously generated using the sequencer and nucleic acid molecules from a selected species; and one or more programs stored in the memory and configured to be executed by the one or more processors, the one or more programs including instructions for: receiving at the one or more processors, from the sequencer, sequencing data for a plurality of nucleic acid molecule colonies comprising nucleic acid molecules derived from the selected species, wherein the sequencing data is generated using a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; selecting, using the one or more processors, sequencing data for a subset of the nucleic acid molecule colonies; calling, using the one or more processors, preliminary sequences for the subset of the nucleic acid molecule colonies, comprising inputting the selected sequencing data into the pre-trained sequencer-specific machine-learning model; mapping, using the one or more processors, the called preliminary sequences to a known reference sequence to identify corresponding reference sequence fragments for the called preliminary sequences; and updating, using the one or more processors, the pre-trained sequencer-specific machine-learning model based on a training data set comprising the selected sequencing data and the corresponding reference sequence fragments.

In some embodiments, the pre-trained sequencer-specific machine-learning model was previously updated based on penultimate sequencing data generated using the same sequencer and penultimate nucleic acid molecules from the same selected species.

In some embodiments, the pre-trained sequencer-specific machine-learning model was previously updated by a method comprising: generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step; selecting penultimate sequencing data for a subset of the penultimate nucleic acid molecule colonies; calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species; mapping the called penultimate preliminary sequences to the known reference sequence to identify corresponding penultimate reference sequence fragments for the called penultimate preliminary sequences; and updating the penultimate pre-trained sequencer-specific machine-learning model based on a penultimate training data set comprising the selected penultimate sequencing data and the corresponding penultimate reference sequence fragments.

In some embodiments, the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

In some embodiments, the pre-trained sequencer-specific machine-learning model was previously initialized using sequencing data previously generated using the same sequencer and nucleic acid molecules from a different selected species.

In some embodiments, the different selected species has a smaller genome than the selected species. In some embodiments, the different selected species is a bacterial species or a viral species. In some embodiments, the different selected species is Escherichia coli.

In some embodiments, the selected species is a primate. In some embodiments, the selected species is a human.

In some embodiments, the sequencer-specific machine-learning model is a neural network. In some embodiments, the sequencer-specific machine-learning model is a convoluted neural network.

In some embodiments, the sequencing data comprises, for each nucleic acid molecule colony, a vector comprising a signal intensity value at each sequencing flow step.

In some embodiments, updating the pre-trained sequencer-specific machine-learning model based on the training data set comprises iteratively updating the pre-trained sequencer-specific machine-learning model using the same training data set until a predetermined quality control threshold is met or surpassed. In some embodiments, the predetermined quality control threshold is a convergence threshold.

In some embodiments, updating the pre-trained sequencer-specific machine-learning model based on the training data set comprises iteratively updating the pre-trained sequencer-specific machine-learning model using the same training data set until a change in a quality control value between iterations is below a predetermined threshold. In some embodiments, the predetermined threshold is a convergence threshold.

In some embodiments, the selected sequencing data for the subset of the nucleic acid molecule colonies is randomly selected.

Also provided herein is a system of any one of the above embodiments, wherein the plurality of nucleic acid molecule colonies comprises a colony comprising the target nucleic acid molecule, and wherein the one or more programs further include instructions for; inputting the sequencing data for the colony comprising the target nucleic acid molecule into the updated sequencer-specific machine-learning model; and calling, for the target nucleic acid molecule, a homopolymer length for each sequencing flow step using the updated sequencer-specific machine-learning model.

Also provided herein is a computer-readable memory storing: a pre-trained sequencer-specific machine-learning model configured to call a homopolymer length or a homopolymer length likelihood for each sequencing flow step based on signal intensity values, wherein the pre-trained sequencer-specific machine-learning model was previously trained based on sequencing data previously generated using a sequencer and nucleic acid molecules from a selected species; and one or more programs comprising instructions, which executed by one or more processors of an electronic device, cause the electronic device to: receive sequencing data for a plurality of nucleic acid molecule colonies comprising nucleic acid molecules derived from the selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; select sequencing data for a subset of the nucleic acid molecule colonies; call preliminary sequences for the subset of the nucleic acid molecule colonies, comprising inputting the selected sequencing data into the pre-trained sequencer-specific machine-learning model; map the called preliminary sequences to a known reference sequence to identify corresponding reference sequence fragments for the called preliminary sequences; and update the pre-trained sequencer-specific machine-learning model based on a training data set comprising the selected sequencing data and the corresponding reference sequence fragments.

In some embodiments, the pre-trained sequencer-specific machine-learning model was previously updated based on penultimate sequencing data generated using the same sequencer and penultimate nucleic acid molecules from the same selected species.

In some embodiments, the pre-trained sequencer-specific machine-learning model was previously updated by a method comprising: generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step; selecting penultimate sequencing data for a subset of the penultimate nucleic acid molecule colonies; calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species; mapping the called penultimate preliminary sequences to the known reference sequence to identify corresponding penultimate reference sequence fragments for the called penultimate preliminary sequences; and updating the penultimate pre-trained sequencer-specific machine-learning model based on a penultimate training data set comprising the selected penultimate sequencing data and the corresponding penultimate reference sequence fragments.

In some embodiments, the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

In some embodiments, the pre-trained sequencer-specific machine-learning model was previously initialized using sequencing data previously generated using the same sequencer and nucleic acid molecules from a different selected species.

In some embodiments, the different selected species has a smaller genome than the selected species. In some embodiments, the different selected species is a bacterial species or a viral species. In some embodiments, the different selected species is Escherichia coli.

In some embodiments, the selected species is a primate. In some embodiments, the selected species is a human.

In some embodiments, the sequencer-specific machine-learning model is a neural network. In some embodiments, the sequencer-specific machine-learning model is a convoluted neural network.

In some embodiments, the sequencing data comprises, for each nucleic acid molecule colony, a vector comprising a signal intensity value at each sequencing flow step.

In some embodiments, updating the pre-trained sequencer-specific machine-learning model based on the training data set comprises iteratively updating the pre-trained sequencer-specific machine-learning model using the same training data set until a predetermined quality control threshold is met or surpassed. In some embodiments, the predetermined quality control threshold is a convergence threshold.

In some embodiments, updating the pre-trained sequencer-specific machine-learning model based on the training data set comprises iteratively updating the pre-trained sequencer-specific machine-learning model using the same training data set until a change in a quality control value between iterations is below a predetermined threshold. In some embodiments, the predetermined threshold is a convergence threshold.

### BRIEF DESCRIPTION OF THE DRAWINGS

The invention will now be described, by way of example only, with reference to the accompanying drawings, in which:

FIG. 1 shows an exemplary method of generating sequencing data for a plurality of nucleic acid molecule colonies using a flow sequencing method, in accordance with some embodiments.

FIG. 2A shows an exemplary flowgram, in accordance with some embodiments.

FIG. 2B shows the exemplary flowgram shown in FIG. 5A with the most likely sequence, given the sequencing data, selected based on the highest likelihood at each flow position (as indicated by stars), in accordance with some embodiments.

FIG. 3A shows a flowchart of an exemplary method of updating a system comprising a sequencer, in accordance with some embodiments.

FIG. 3B shows a flowchart of an exemplary method of obtaining training data (A in FIG. 3A), in accordance with some embodiments.

FIG. 4 shows a surface/support sequencer schematic, in accordance with some embodiments.

FIG. 5 shows exemplary data collection from n flow steps and exemplary data structure corresponding to an individual nucleic acid colony, in accordance with some embodiments.

FIG. 6 shows a schematic of a called preliminary sequence to a mapped sequence, in accordance with some embodiments.

FIG. 7A shows an example of a series of sequencing runs, beginning with an initialization model through the current model. The figure further illustrates one method of updating the current model, in accordance with some embodiments.

FIG. 7B shows an example of a series of sequencing runs, beginning with an initialization model through the current model. The figure further illustrates one method of updating the current model, in accordance with some embodiments.

FIG. 8A shows an example of a computing device in accordance with some embodiments, which may be used to implement a method as described herein, in accordance with some embodiments.

FIG. 8B shows an exemplary block diagram of a sequencing read data set, in accordance with some embodiments.

FIG. 8C shows an exemplary block diagram of a sequencing read data set, in accordance with some embodiments.

FIG. 9 shows the model convergence comparison between a traditional model and an adaptive-based model for use in base calling, in accordance with some embodiments.

### DETAILED DESCRIPTION OF THE INVENTION

Described herein are methods for updating a system comprising a nucleic acid molecule sequencer to account for instrument drift of the sequencer over time (e.g., to calibrate the system or recalibrate the system). Instrument drift refers to changes in the operation of an instrument that often occur gradually, but predictably, and which can threaten the validity of conclusions drawn from the data obtained with that instrument over time. Instrument drift affects signal detection, and thus the overall accuracy of the sequencing readout. Instrument drift presents a particular problem in base calling homopolymer lengths, for example, in the context of a flow sequencing method, because the homopolymer length call is based on signal intensity and instrument drift can cause an inaccurate interpretation of the signal intensity. Periodic recalibration of the instrument can help to minimize instrument drift.

Sequencing data for a plurality of nucleic acid molecule colonies comprising nucleic acid molecules derived from a selected species may be generated using a flow sequencing method. For example, the sequencing data may be generated by extending sequencing primers hybridized to nucleic acid molecules using a plurality of sequencing flow steps. Each sequencing flow step includes substeps, including (i) combining the plurality of nucleic acid molecule colonies with nucleotides, wherein at least a portion of the nucleotides are labeled, and (ii) measuring, for each nucleic acid molecule colony, a signal intensity value indicating nucleotide incorporation into the sequencing primers hybridized to said nucleic acid molecules. The sequencing data can therefore include, for each nucleic acid molecule colony, a signal intensity value at each sequencing flow step.

In some embodiments, the nucleic acid sequencer relies on a trained machine-learning model to interpret signal intensity. For example, the model is configured to receive a signal intensity value indicative of nucleotide incorporation into a sequencing primer (e.g., measured for each sequencing flow step of a flow sequencing method) and determine a homopolymer length or a homopolymer length likelihood as its output. The machine-learning model can be specific to the sequencer (e.g., trained using sequencer-specific data) because each sequencer can have independent variances. Instrument drift can cause inaccurate outputs of a machine-learning model trained using data from multiple sequences because the drift in each instrument may result in independent deviations in the performance of the measuring system over time. Instrument drift can be caused by a variety of factors, including, but not limited to, the age of the machine and its components, the usage patterns of the machine, and the ambient conditions (e.g., temperature, humidity, etc.) surrounding the machine.

To compensate for this instrument drift and to ensure accurate sequencing output, one solution is to generate de novo models regularly. An initial sequencer-specific machine-learning model may be built de novo, for example as described in WO 2020/185790. While this method allows for accurate homopolymer length calls, de novo model generation is time consuming and can exceed the time needed to collect sequencing data for a particular sequencing run. Thus, processing the sequencing data to accurately call a sequence, including generating a de novo model, can result in a backlog of sequencing data from various sequencing runs to be processed. A more efficient method of processing the sequencing data that includes system calibration is needed to address the sequencing data backlog, while also accounting for instrument drift.

Embodiments of the present disclosure include efficiently recalibrating the nucleic acid sequencer at regular intervals, such as for each sequencing run. In some embodiments, the recalibration method can include updating (e.g., retraining) the machine-learning model at regular intervals. Retraining a trained model can be less time-consuming than generating a de novo model and can require less training data, thus improving memory usage and management. Further, such models can require less processing power for training and for performing the trained tasks. Thus, embodiments of the present disclosure can improve the functioning of a computer system by improving processing speed and allowing for efficient use of computer memory and processing power.

In some embodiments, the sequencer is associated with multiple machine-learning models, and the recalibration method includes selecting a model from the multiple machine-learning models to recalibrate. The sequencer-specific machine-learning model can be recalibrated using sequencing data received from the same sequencer in any of the previous sequencing runs. In some implementations of the method, the pre-trained sequencer-specific machine-learning model selected to be recalibrated (e.g., the current model) is a machine-learning model trained for the same sequencer on the data from an immediately prior (i.e., penultimate) sequencing run. In other implementations of the method, the pre-trained sequencer-specific machine-learning model selected to be recalibrated is a machine-learning model trained for the same sequencer on the data from some prior sequencing run, and the machine-learning model is selected from a plurality of prior sequencing runs based on some threshold, which, in some examples, may be indicative of higher predictive quality (e.g., as compared with other available pre-trained sequencer-specific machine learning models trained for the same sequencer on data from other prior sequencing runs).

A portion of sequencing data generated from a particular sequencing run can be used to update a pre-trained sequencer-specific machine-learning model. To update the model, the sequencing data is received (e.g., by one or more processors), and a subset of the sequencing data may be selected to update the system. Preliminary sequences for the selected subset of sequencing data are called using a pre-trained machine-learning model that has been configured to call homopolymer lengths or homopolymer length likelihoods for each sequencing flow step based on the signal intensity values. The preliminary sequences are then mapped to known reference sequences to identify corresponding reference sequence fragments for the called preliminary sequences. The identified corresponding reference sequence fragments can operate as a ground truth for use in updating the system. The pre-trained sequencer-specific machine-learning model can then be updated using a training data set that includes the selected sequencing data and the identified corresponding reference sequence fragments.

Updating the system comprising a sequencer can include: (a) receiving, at one or more processors, sequencing data for a plurality of nucleic acid molecule colonies comprising nucleic acid molecules derived from a selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; (b) selecting, using the one or more processors, sequencing data for a subset of the nucleic acid molecule colonies; (c) calling, using the one or more processors, preliminary sequences for the subset of the nucleic acid molecule colonies, comprising inputting the selected sequencing data into pre-trained sequencer-specific machine-learning model configured to call a homopolymer length or a homopolymer length likelihood for each sequencing flow step based on the signal intensity values, wherein the pre-trained sequencer-specific machine-learning model was previously trained based on sequencing data previously generated using the same sequencer and nucleic acid molecules from the same selected species; (d) mapping, using the one or more processors the called preliminary sequences to a known reference sequence to identify corresponding reference sequence fragments for the called preliminary sequences; and (e) updating, using the one or more processors the pre-trained sequencer-specific machine-learning model based on a training data set comprising the selected sequencing data and the corresponding reference sequence fragments. The updated sequencer-specific machine-learning model may subsequently be used to call a sequence for the sequencing data (e.g., the full sequencing data set).

The methods described herein may be computer-implemented methods, and one or more steps of the method may be performed, for example, using one or more computer processors.

Also provided herein is a system comprising a sequencer, one or more processors, a computer-readable memory, and one or more programs, the one or more programs comprising instructions, which when executed by one or more processors of an electronic device, cause the electronic device to perform any one or more of the methods described herein.

Further provided herein is a non-transitory computer-readable storage medium storing one or more programs, the one or more programs comprising instructions, which when executed by one or more processors of an electronic device, cause the electronic device to perform any one or more of the methods described herein.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

A “flow order” refers to the order of separate nucleotide flows used to sequence a nucleic acid molecule using non-terminating nucleotides. A flow order may have any number of nucleotide flows. A flow order may be expressed as a one-dimensional matrix or linear array of bases corresponding to the identities of, and arranged in chronological order of, the nucleotide flows provided to the sequencing reaction space: (e.g., [A-T-G-C-A-T-G-C-A-T-G-A-T-G-A-T-G-A-T-G-C-A-T-G-C]). Such a one-dimensional matrix or linear array of bases in the flow order may also be referred to herein as a “flow space.” Each entry in flow space (e.g., each element in the one-dimensional matrix or linear array) indicates a flow position. A “flow position” refers to the sequential position of a given separate nucleotide flow during the sequencing process. The flow order may be divided into cycles of repeating units (i.e., a “flow cycle”), and the flow order of the repeating units is termed a “flow-cycle order.” A flow cycle may be expressed as a one-dimensional matrix or linear array of an order of bases corresponding to the identities of, and arranged in chronological order of, the nucleotide flows provided within the sub-group of contiguous flow(s) (e.g., [A-T-G-C], [A-A-T-T-G-G-C-C], [A-T], [A/T-A/G], [A-A], [A], [A-T-G], etc.). A flow cycle may have any number of nucleotide flows. A given flow cycle may be repeated one or more times in the flow cycle, consecutively or non-consecutively. For example, where [A-T-G-C] is identified as a 1st flow cycle, and [A T G] is identified as a 2nd flow cycle, the flow order of [A-T-G-C-A-T-G-C-A-T-G-A-T-G-A-T-G-A-T-G-C-A-T-G-C] may be described as having a flow-cycle order of [1st flow cycle; 1st flow cycle; 2nd flow cycle; 2nd flow cycle; 2nd flow cycle; 1st flow cycle; 1st flow cycle]. Alternatively or in addition, the flow-cycle order may be described as [cycle 1, cycle 2, cycle 3, cycle 4, cycle 5, cycle 6], where cycle 1 would be the 1st flow order, cycle 2 would be the 1st flow order, cycle 3 would be the 2nd flow cycle order, etc.

The term “homopolymer length” refers to a number of sequential identical nucleotides of a particular base type in a nucleic acid sequence at a given flow step. The homopolymer length may be 0, 1, 2, 3 or any other 0 or positive integer value. A “homopolymer length likelihood” refers to a statistical parameter indicative of a likelihood or confidence interval that a given homopolymer length at a particular flow step is the correct homopolymer length.

The terms “individual,” “patient,” and “subject” are used synonymously, and refer to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. Alternatively or in addition, a subject may be known to have previously had a disease or disorder. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. The biological sample can be a fluid, tissue, collection of cells (e.g., check swab), hair sample, or feces sample. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. Alternatively, the sample may be obtained from any other source including but not limited to blood, sweat, hair follicle, buccal tissue, tears, menses, feces, or saliva of a subject. The biological sample may be a tissue sample, such as a tumor biopsy. The tissue can be from an organ (e.g., liver, lung, or thyroid), or a mass of cellular material, such as, for example, a tumor. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or in addition, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

The term “label,” as used herein, refers to a detectable moiety that is coupled to or may be coupled to another moiety, for example, a nucleotide or nucleotide analog. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease). In some embodiments, the label is a fluorophore.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide). The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqucosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). A “non-terminating nucleotide” is a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “nucleotide flow” refers to a set of one or more non-terminating nucleotides (which may be labeled or a portion of which may be labeled). The nucleotide flow may be provided to a sequencing reaction space in a temporally distinct instance of providing a nucleotide-containing reagent. For example, providing two flows may refer to (i) providing a nucleotide-containing reagent (e.g., an A-base containing solution) to a sequencing reaction space at a first time point and (ii) providing a nucleotide-containing reagent (e.g., a G-base containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), e.g., 1, 2, 3, or 4 canonical base types.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof.

Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s).

The terms “reference genome” and “reference sequence,” as used herein, generally refer to a standardized genomic sequence or a portion thereof (e.g., any genome known in the art). In some embodiments, a reference sequence comprises a reference genome or a portion of reference genome (e.g., for a same species as a subject from which a biological sample was taken for analysis). A reference genome may be a representative example of a set of genes. In some instances, a reference genome is generalized to a species (e.g., Homo sapiens) and is determined from one or more assembled or partially assembled genome sequences of one or more individuals of said species. In some instances, a reference genome is specific to an individual of a species, and in such instances the reference genome may be determined from one or more assembled or partially assembled genome sequences from said individual. In some embodiments, a reference genome refers to any known genome of an organism or virus (e.g., a genome that is partially or completely assembled) that may be used for alignment of sequences from a subject. A reference genome may be any portion of a genomic nucleic acid sequence (e.g., a targeted panel of genes, one or more chromosomes, an entire genome of a species, etc.) that is used as a comparison for generated nucleic acid sequencing data (e.g., sequencing information generated according to sequencing methods described herein). Examples of human reference genomes include NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). Example human reference genomes can be accessed from online genome browsers hosted by either the National Center for Biotechnology Information (NCBI) or the University of California, Santa Cruz (UCSC).

The term “sequencing,” as used herein, generally refers to a process for generating or identifying a sequence of a biological molecule, such as a nucleic molecule. Such sequence may be a nucleic acid sequence, which may include a sequence of nucleic acid bases. Sequencing may be single molecule sequencing or sequencing by synthesis, for example. Sequencing may be performed using template nucleic acid molecules immobilized on a support, such as a flow cell or one or more beads on a substrate as described herein. Examples of sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Generating Sequencing Data Using Flow Sequencing Methods

Sequencing data can be generated using a flow sequencing method that includes extending a primer bound to a template nucleic acid molecule according to a pre-determined flow cycle or flow order where, in any given flow position, a type of nucleotide base is accessible to the extending primer. More commonly, a single type of nucleotide base is used in any given sequencing flow, although in some variations, two or three different types of nucleotide bases may be used, which allows for a faster primer extension but may provide less sequencing data about the sequence region. In some embodiments, at least some of the nucleotides of the particular type include a label, which upon incorporation of the labeled nucleotides into the extending primer renders a detectable signal. The resulting sequence by which such nucleotides are incorporated into the extended primer should be the reverse complement of the sequence of the template nucleic acid molecule. For example, sequencing data may be generated using a flow sequencing method that includes extending a primer using labeled nucleotides, and detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473, International patent application WO 2021/007495 A1, International patent application WO 2020/227143 A1, and International patent application WO 2020/227137 A1, which are each incorporated herein by reference in their entirety. While the following description is provided in reference to flow sequencing methods, it is understood that other sequencing methods may be used to sequence all or a portion of the sequenced region.

Flow sequencing includes the use of nucleotides to extend the primer hybridized to the nucleic acid molecule. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. Alternative orders may be readily contemplated by one skilled in the art. In some instances, the order of any cycle may be any permutation of the nucleotides A, G, C, and T (or U). Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase, Bst 3.0 DNA polymerase, Bsu DNA polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template nucleic acid molecule can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

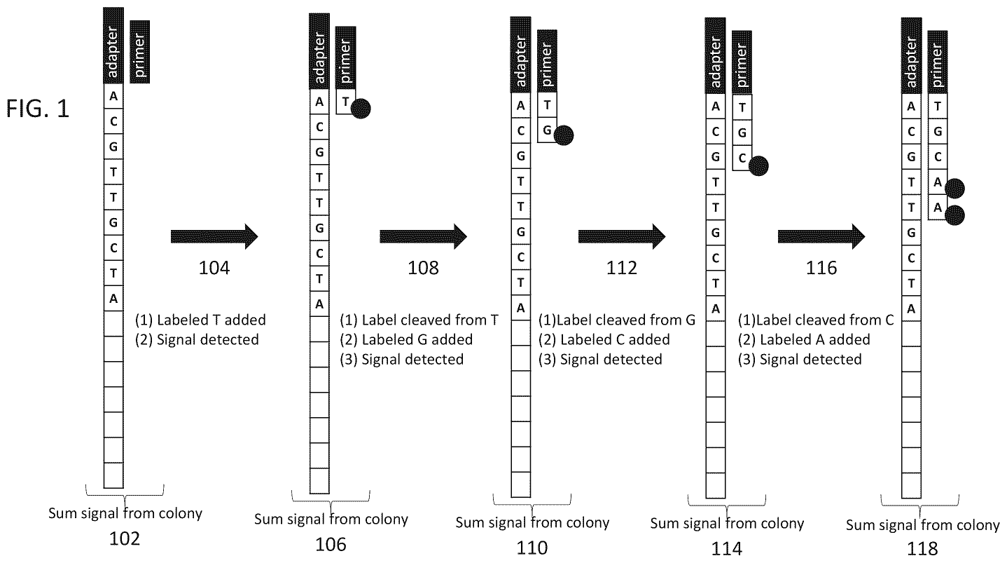

FIG. 1 illustrates an exemplary flow sequencing method that may be used to generate the sequencing data described herein. Polynucleotides may be bound to a surface (for example, a bead, which is optionally itself tethered to another surface). The surface-bound polynucleotides may be amplified to form sequencing colonies on the surface. The polynucleotides include the nucleic acid sequence of interest (e.g., a nucleic acid molecule from or derived from a subject), and can further include a sequencing adapter sequence. The adapter sequence can include a sequencing primer hybridization site. As shown at 102, a sequencing primer is hybridized to the adapter sequence of the polynucleotide at the sequencing primer hybridization site. The sequencing primer is then extended using a series of flow steps, which include combining the hybrid DNA molecule (i.e., the polynucleotide hybridized to the sequencing primer) with nucleotides, at least a portion of which are labeled, followed by the detection of a signal from the labeled nucleotides. Detected signals indicate nucleotide incorporation into the sequencing primer. The sequencing colonies may be washed with a wash buffer to remove unincorporated nucleotides prior to signal detection. The signal may be detected, for example, by imaging the surface. The intensity of the signal is indicative of how many labeled nucleotides were incorporated into the sequencing primer, summed across the colony. In the example shown in FIG. 1, nucleotides are added in four flow steps, with a single type of nucleobase being combined with the hybrid DNA molecules in any given flow step according to the cycle T-G-C-A. At 104, labeled T nucleotides are combined with the hybrid. Since the T base is complementary to the A base in the template polynucleotide it is incorporated into the extending primer to form the hybrid DNA molecule in 106. The signal from the labeled T nucleotide that is incorporated into the sequencing primer is then detected. Since the colonies include identical copies of the same polynucleotide (except that in some cases a rare error—i.e., an incorrect nucleotide—may be incorporated during amplification), the signal that is detected is the sum signal from the colony. Thus, the amount of labeled T nucleotide compared with unlabeled T nucleotide may be calibrated such that the signal is accurately detected within the range of the signal detection equipment (e.g., a camera or other sensor). After detecting the signal intensity, the label may be removed from the T nucleotide, for example by cleaving or excising the label from the nucleotide, at 108. The sequencing method can then be continued with the next base in the flow order, G in the example illustrated in FIG. 1. At 108, labeled G nucleotides are combined with the hybrid. Since the G base is complementary to the C base in the template polynucleotide it is incorporated into the extending primer to form the hybrid in 110. The signal from the labeled G nucleotide incorporated into the sequencing primer is then detected. The label may then be removed from the G nucleotide at 112 before labeled C nucleotides are combined with the hybrid DNA molecule, and a signal indicative of C nucleotide incorporation into the sequencing primer is detected. More particularly, since C is complementary to the G base in the template polynucleotide it is incorporated into the extending primer to form the hybrid DNA molecule at 114. The label may then be removed from the C nucleotide at 116 before labeled A nucleotides are combined with the hybrid DNA molecule. Since the A nucleotide is complementary to the T nucleotides in the template strand the labeled A nucleotide will be incorporated into the extending sequencing primer to form the hybrid DNA molecule at 118. Further, because the template strand includes two consecutive T bases, two A nucleotides are incorporated into the extending sequencing primer. Non-consecutive T bases later in the template strand will not lead to the incorporation of A nucleotides in this flow step. Importantly, the detected signal intensity indicating the incorporation of two A nucleotides will be greater than the signal intensity indicating the incorporation of one nucleotide. In some flow steps, no nucleotide base may be incorporated into the sequencing primer (for example, in the absence of a complementary bases in the template polynucleotide), and in such flow steps no signal will be detected. In some flow steps, more than two nucleotides may be incorporated into the sequencing primer, and in such flow steps the detected signal will be greater than the signal intensity indicating the incorporation of one or two nucleotides. In some cases, the signal intensity will be proportional or approximately proportional to the number of nucleotides incorporated into the sequencing primer.

Primer extension using flow sequencing allows for long-range sequencing on the order of hundreds or even thousands of bases in length. The number of flow steps or cycles can be increased or decreased to obtain the desired sequencing length. Extension of the sequencing primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

The sequencing data set is uniquely structured to provide a computationally efficient analysis. The sequencing data set for the nucleic acid molecule colonies can include flow signals at flow positions that each corresponds to a flow of a particular nucleotide. Using this uniquely structured data set, the nucleic acid molecule (or molecules) can be analyzed in “flow space” rather than “base space” (also referred to as “nucleotide space” or “sequence space”). The flow space data depend on additional information related to the flow-cycle order, which is not carried by base space data. See, e.g., International published application WO 2020/227137 A1.

The resulting sequencing data for each colony includes a measured signal intensity at each individual flow step. The sequencing data can be received by one or more processors in a computer-implemented method. In some embodiments, the sequencing data is stored in a non-transitory computer-readable medium that is accessible by the one or more processors. The sequencing data may include, for example, a vector comprising a signal intensity value at each sequencing flow step for each nucleic acid molecule colony. Accordingly, each nucleic acid molecule colony may be assigned a vector comprising a 1×n matrix (i.e., an n-dimensional vector), where n=the number of flow steps, and where each component of the vector is the signal intensity recorded at that individual flow step for that particular nucleic acid molecule colony.

Prior to generating the sequencing data, sequencing colonies can be formed. The nucleic acid molecules sequenced according to the methods described herein may be obtained from a selected species from any suitable biological source (e.g., biological sample). The selected species may be a vertebrate, such as a mammal. In some embodiments, the selected species is a primate, a dog, a cat, a rodent (e.g., a rat, mouse, etc.), pig, sheep, cow, etc. In some embodiments, the selected species is a human. The nucleic acid molecules from the selected species may be obtained from, for example a tissue sample (e.g., a tumor biopsy), a blood sample, a plasma sample, a saliva sample, a fecal sample, or a urine sample. The nucleic acid molecules may be DNA or RNA polynucleotides. In some embodiments, RNA polynucleotides are reverse transcribed into DNA polynucleotides prior to hybridizing the polynucleotide to the sequencing primer. In some embodiments, the polynucleotide is a cell-free DNA (cfDNA), such as a circulating tumor DNA (ctDNA) or a fetal cell-free DNA. The nucleic acid molecules may be randomly fragmented, for example in vivo (e.g., as in cfDNA) or in vitro (for example, by sonication or enzymatic fragmentation).

Sequencing libraries of the nucleic acid molecules may be prepared through known methods. The nucleic acid molecules may be ligated to an adapter sequence. The adapter sequence may include a hybridization sequence that hybridized to the primer extended during the generated of the coupled sequencing read pair. For example, the hybridization sequence of the adapter may be a uniform sequence across a plurality of different nucleic acid molecules, and the sequencing primer may be a uniform sequencing primer. This allows for multiplexed sequencing of different nucleic acid molecules in a sequencing library. Optionally, the adapter sequence includes one or more barcode regions and/or unique molecular identifiers (UMIs). The nucleic acid molecule may be ligated to an adapter during sequencing library preparation.

The nucleic acid molecule may be attached to a surface (such as a solid support) for sequencing. The solid support may be a bead, which may be attached to a wafer. The wafer may be an annulus-shaped (i.e., disc-shaped with a central hole) surface comprised of concentric rings. Each ring may be comprised of individual tiles to which the nucleic acid-bead conjugates are attached. In some versions of generating sequencing data, the bead may first be attached to the wafer, then the nucleic acid may be attached to the bead. In other versions of generating sequencing data, the nucleic acid may first be attached to the bead and the nucleic acid-bead conjugate may then be attached to the wafer.

The nucleic acid molecules may be amplified (for example, by bridge amplification or other amplification techniques) to generate nucleic acid molecule sequencing colonies. The amplified nucleic acid molecules within the cluster are substantially identical or complementary (some errors may be introduced during the amplification process such that a portion of the nucleic acid molecules may not necessarily be identical to the original nucleic acid molecules). Colony formation allows for signal amplification so that the detector can accurately detect incorporation of labeled nucleotides for each colony. Colony amplification is not a perfect process, though, and errors can be introduced at this stage. Any errors that occur during the amplification step can result in additional background signal noise, but the generation of colonies with many identical, amplified template nucleic acid molecules per bead decreases the impact that any individual amplification error might have on the overall quality of the signal intensity and subsequent sequencing output data for any single sequencing colony. In some cases, the colony is formed on a bead using emulsion PCR and the beads are distributed over a sequencing surface. Examples for systems and methods for sequencing can be found in U.S. Pat. No. 10,344,328 and International patent application WO 2020/227143, each of which is incorporated herein by reference in its entirety.

### Calibrating or Recalibrating the System

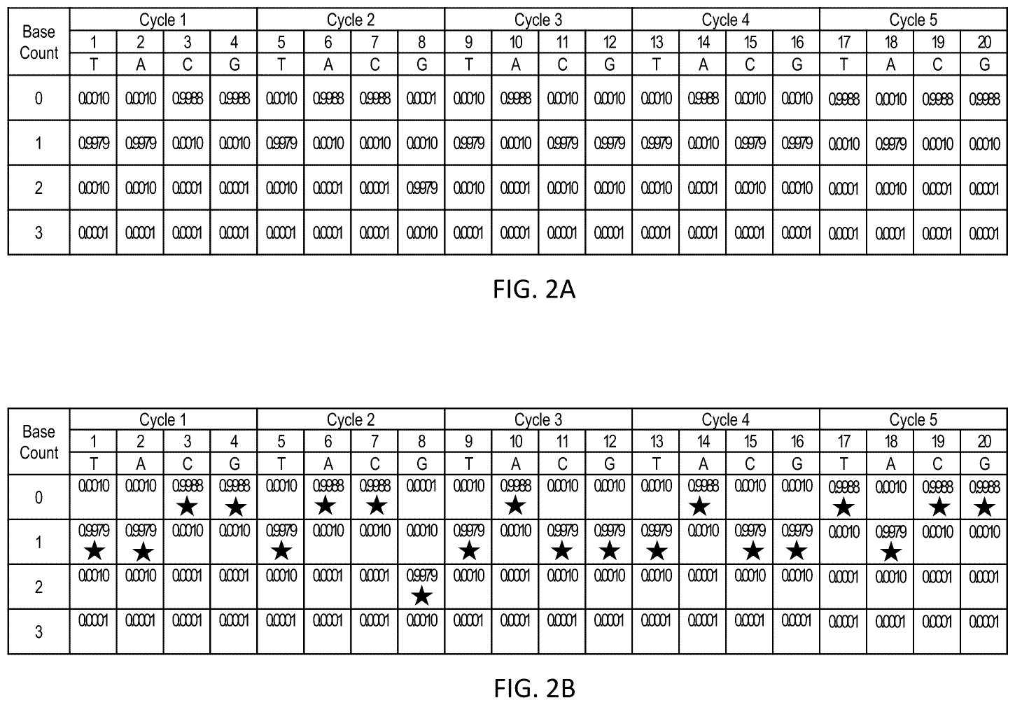

The flow sequencing method described herein can rely on a machine-learning model to update a system so that it accurately calls sequences more quickly and efficiently than using de novo initialization of the model. For example, with reference to FIG. 1, after each flow step (e.g., 104, 106, 110, 114, or 118), a signal intensity indicative of nucleotide incorporation into a sequencing primer is measured. The signal intensity can be fed into a trained machine-learning model, which outputs a homopolymer length or a homopolymer length likelihood as its output (e.g., each column in FIG. 2A is for an individual flow step).

As discussed above, instrument drift can cause inaccurate output of machine-learning models over repeated sequencing runs (e.g., due in part to inaccurate tracking of sequencing colonies over time and over multiple flow steps and/or flow cycles). Instrument drift can be caused by a variety of factors, including the age of the machine and ambient conditions of the machine (e.g., the temperature or humidity of the surrounding environment). Thus, a method is needed to efficiently recalibrate the system during the flow sequencing method. Specifically, a method is needed to recalibrate the machine-learning model during and between implementations of flow sequencing methods.

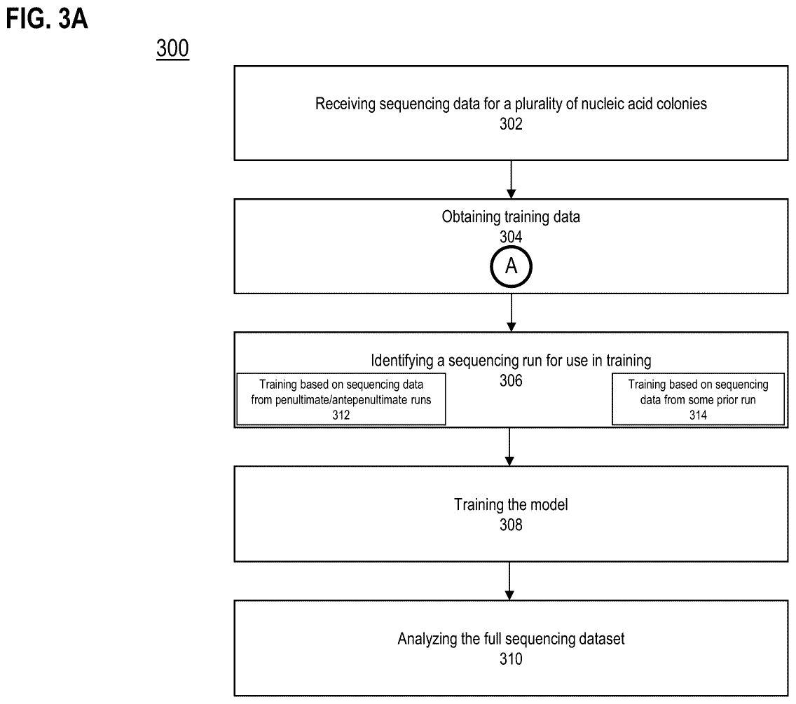

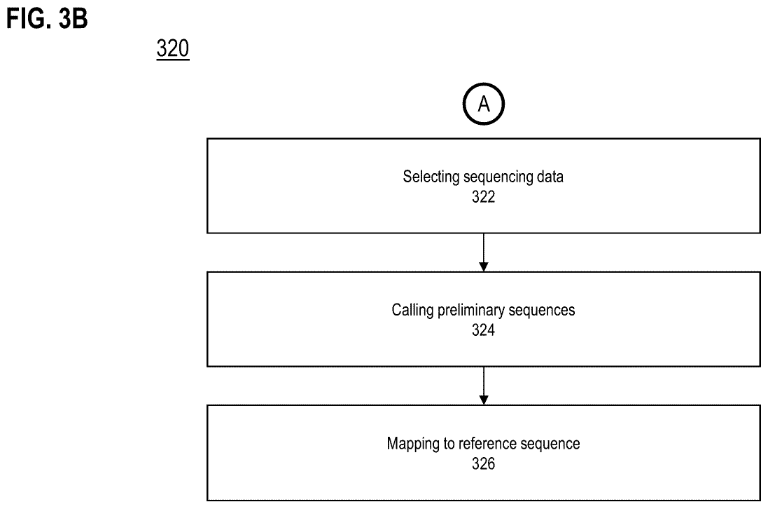

FIG. 3A shows an exemplary method 300 for updating a system comprising a sequencer. In some embodiments, this method is performed after a plurality of flow steps, where each flow step represents the introduction of a nucleotide or nucleotides, at least a portion of which are labeled. The method of updating a system may be performed once or at regular intervals (e.g., after each sequencing run or after a plurality of sequencing runs). The full sequencing dataset may be generated or received at step 302 (FIG. 3A). The full data set can include flow sequencing data for a plurality of colonies. For each colony, the flow sequencing data include a signal intensity value for each flow step. A training set may be obtained from the received or generated dataset at step 304 (FIG. 3A), as described below. The selected dataset set is a subset of the full dataset, and each colony can be represented by a vector. In some embodiments, the training set may be obtained as in process 320 (FIG. 3B; illustrated as A in FIG. 3A). With reference to FIG. 3B, a subset of sequencing data may be selected at step 322. Preliminary sequences of the subset of sequencing data may then be called at step 324. The preliminary sequences that may be generated at step 324 may then be mapped to a known reference sequence (e.g., from a reference genome) at step 326. The mapped preliminary sequence/reference sequence pair may function as a training data pair to iteratively train a model until convergence of the model is achieved.

With reference to FIG. 3A, a decision may be made at step 306 whether to train the model based on sequencing data (i.e., step 312) from penultimate/antepenultimate runs or on sequencing data (i.e., step 314) from some prior run selected, for example, for high quality of the data. At step 308, the model can then be trained using the training data. Once the model is trained, the full sequencing data set can be trained using the trained model (see step 310, FIG. 3A).

At step 302, sequencing data for nucleic acid molecule colonies are received, for example by one or more processors. The data generated or received at step 302 is sequencing data produced by a sequencer and may be collected after a series of flow steps, where each flow step represents the introduction of a nucleotide or nucleotides, at least a portion of which are labeled. The full data set can include flow sequencing data for a plurality of colonies. For each colony, the flow sequencing data includes the signal intensity values for each flow step.

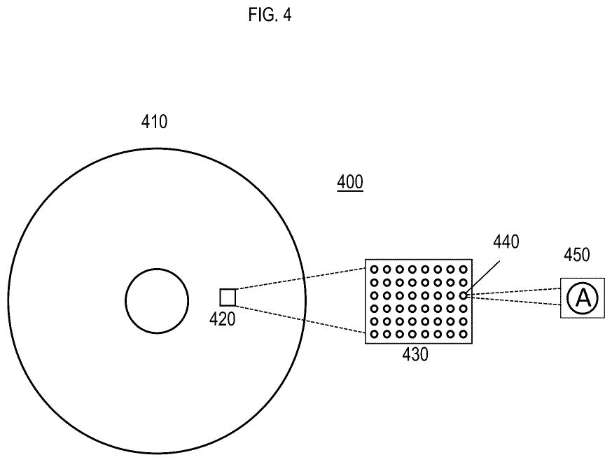

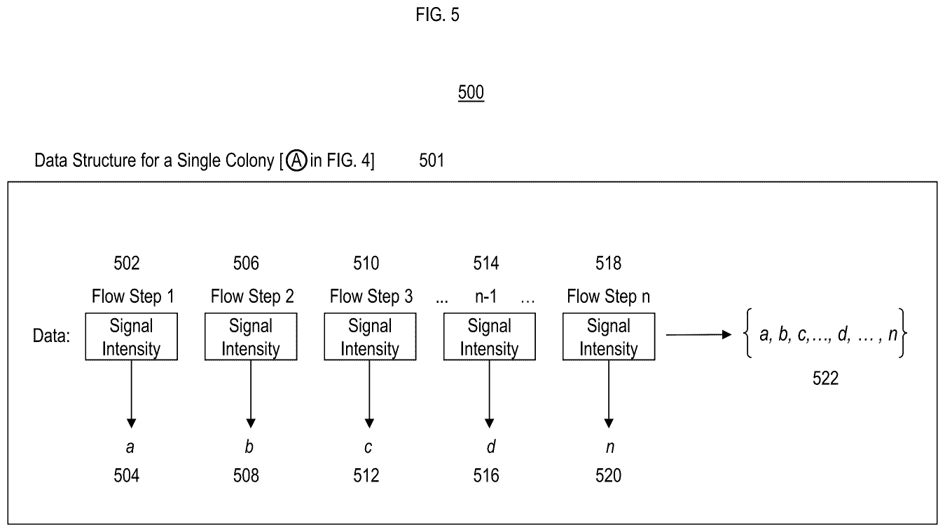

The sequencing data of the nucleic acid molecule colonies that include a plurality of copies of a nucleic acid molecule from a selected species may be received or generated from a sequencer comprising a surface (e.g., a wafer) as illustrated in FIG. 4 (schematic 400). The nucleic acid molecules may be attached to a surface (e.g., a bead, a flowcell, a wafer, etc.) and amplified to form the colonies. The surface may be a wafer, which may be an annulus-shaped surface comprised of concentric rings. Each ring may be comprised of individual tiles (e.g., tile 420). Nucleic acids may be attached to a solid support, which may be a bead, which may be attached to the wafer. Each nucleic acid-support conjugate, which may be a nucleic acid-bead conjugate, may comprise a nucleic acid colony (e.g., individually addressable locations 440). An individual tile (e.g., tile 420) may be comprised of several nucleic acid-support conjugates, as illustrated in 430.

The sequencing data can be generated using a flow sequencing method, for example by extending sequencing primers hybridized to the nucleic acid molecules using a plurality of sequencing flow steps. The sequencing flow steps are performed by combining the colonies with nucleotides (at least a portion of which are labeled), and measuring, for each colony, a signal intensity value indicating nucleotide incorporation into the sequencing primers. The sequencing data includes, for each colony, a signal intensity value at each flow step.

For example, for each individual nucleic acid colony (illustrated as ‘A’ 450 in FIG. 4 and reflected as ‘A’ 501 in FIG. 5) a series of data may be collected (FIG. 5). For an individual colony, a signal intensity may be collected after each flow step, as illustrated in exemplary method 500 in FIG. 5. For an individual colony, e.g., colony 501, a first flow step 502 may occur. After the introduction of the nucleotide or nucleotides from the first flow step 502, a signal intensity may be recorded for each colony (e.g., a at 504). After the signal intensity is recorded, a second flow step 506 may occur. After the introduction of the nucleotide or nucleotides from the second flow step 506, a signal intensity may be recorded for each colony (e.g., b at 508). After the signal intensity is recorded, a third flow step 510 may occur. After the introduction of the nucleotide or nucleotides from the third flow step 510, a signal intensity may be recorded for each colony (e.g., c at 512). After the signal intensity is recorded, an n−1 flow step 514 may occur. After the introduction of the nucleotide or nucleotides from the n−1 flow step, a signal intensity may be recorded for each colony (e.g., d at 516). After the signal intensity is recorded, an n flow step 518 may occur. After the introduction of the nucleotide or nucleotides from the n flow step, a signal intensity may be recorded for each colony (e.g., n at 520). The recorded signal intensity for a given colony (e.g., colony 501) can then be arranged into a 1×n matrix 522, where the signal intensity for each flow step is recorded as an individual element (e.g., values a, b, c, . . . , d, . . . , n). A matrix containing the signal intensity data each colony for each flow step can then be collected and may comprise the full received sequencing dataset. For example, for each of the colonies in 430, a 1×n matrix, as described above, may be collected where each matrix element represents the signal intensity for each flow step. The collection (i.e., array) of 1×n matrices represents the full generated or received sequencing data set at step 302.

At step 304, training data are obtained. The training data may be obtained as in process 320 (FIG. 3B; illustrated as A in FIG. 3A). A subset of sequencing data may be selected at step 322 (FIG. 3B). The subset of sequencing data is selected from the full data set that may be received at step 302. The full dataset may be comprised of a 1×n matrix for each colony, where each component of the matrix is the signal intensity for an individual flow step, as described above and in FIG. 4 and FIG. 5. A subset of the full data set received at step 302 is selected for generating a training set. The selected subset of colony vectors (e.g., 1×n matrices) from the full sequencing data set may be selected randomly, manually, or through an automated procedure. Random selection minimizes bias when generating the training set. The selected subset may be structured similarly to the full data set. The selected sequencing may be less than about 10% of the generated sequencing data set, such as about 9% or less, about 8% or less, about 7% or less, about 6% or less, about 5% or less, about 4% or less, about 3% or less, about 2% or less, or about 1% or less of the generated sequencing data. The selected subset may also be much less than about 10% of the received or generated sequencing data set, such as about 1% or less, about 0.5% or less, about 0.25% or less, about 0.125% or less, about 0.0625% or less, about 0.03% or less, about 0.02% or less, about 0.01% or less, about 0.001% or less, or about 0.0001% or less of the generated or received sequencing data.

At step 324 (FIG. 3B), preliminary sequences for the subset of the nucleic acid molecule colonies may be called using the selected subset of sequencing data. For each colony vector in the subset, a corresponding preliminary sequence can be obtained. A preliminary sequence from the sequencing data may be called without a sequence alignment. For each of the 1×n matrices, the most likely sequence (e.g., a preliminary sequence), given the data, can be determined by selecting the base count with the highest likelihood at each flow position, as shown by the stars in FIG. 2B. The sequence of the primer extension can be determined according to the most likely base at each flow position. The preliminary sequence can then be used to generate a training data set at step 304 (FIG. 3A; see also, FIG. 3B).

Preliminary sequences for the colonies can be called using the selected subset of sequencing data. To call the preliminary sequences, the selected sequencing data (e.g., a vector comprising the signal intensity value at each flow step for each of the selected colonies) are input into a pre-trained sequencer-specific machine-learning model that has been configured to call a homopolymer length or a homopolymer length likelihood for each sequencing flow step based on signal intensity values. An exemplary machine-learning model configured to call a homopolymer length for each sequencing flow step based on signal intensity values is described in published International application WO 2019/084158. Importantly, this pre-trained machine-learning model was been previously trained based on sequencing data generated using the same sequencer and nucleic acid molecules from the same selected species. The output of the machine-learning model is a preliminary sequence (e.g., representing the homopolymer length and the homopolymer length likelihood for each flow step, e.g., the likelihood that 0, 1, 2, 3, etc. nucleotides were incorporated). In some implementations of the method, the preliminary sequence is outputted from the machine-learning model as a preliminary sequence in base space (i.e., a sequential presentation of nucleotide bases). In some implementations of the method, the preliminary sequence is outputted from the machine-learning model as a preliminary sequence in flow space. A preliminary sequence may be presented in flow space, for example, using a flowgram. Sequences reported in base space and sequences reported in flow space are interconvertible, as long as the flow cycle (i.e., the order the nucleotides were added to the sequencing reaction) is known.

A flowgram includes information about a homopolymer length at any given flow step according to the flow sequencing method. Take, for example, the flowing template sequences: CTG and CAG, and a repeating flow cycle of T-A-C-G (that is, sequential addition of T, A, C, and G nucleotides, which would be incorporated into the primer only if a complementary base is present in the template nucleic acid molecule). An exemplary resulting flowgram (e.g., with respective rows representing flowgrams for each indicated sequence, CTG, CAG, and CCG) is shown in Table 1, where 1 indicates incorporation of an introduced nucleotide, 2 indicates incorporation of 2 introduced nucleotides of a same type, and 0 indicates no incorporation of an introduced nucleotide. The flowgram can be used to determine the sequence of the template strand.

TABLE 1

| | Cycle 1 | | | | Cycle 2 | | | |
|---|---|---|---|---|---|---|---|---|
| Flow Position | 1 | 2 | 3 | 4 | 5 | 6 | 7 | 8 |
| Nucleotide Flow | T | A | C | G | T | A | C | G |
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

Flowgrams can be used to quantitatively determine a number of incorporated nucleotide from each stepwise introduction. For example, a sequence of CCG would incorporate two G bases, and any signal emitted by the labeled base in that flow cycle would have a greater intensity than the incorporation of a single base. The resulting signals from using a T-A-C-G flow order to sequence three different sequences are shown in Table 1. The flowgram may provide an integer number of bases of the particular type (i.e., a homopolymer length) at each flow position, as shown in Table 1.

Alternatively or in addition, a flowgram can provide one or more homopolymer length likelihoods. The homopolymer length likelihood may be a statistical likelihood in some embodiments. The flow signal is determined from an analog signal that is detected during the sequencing process, such as a fluorescent signal of the one or more bases incorporated into the sequencing primer during sequencing. Although an integer number of zero or more bases are incorporated at any given flow position, a given analog signal many not perfectly match with the analog signal. Therefore, given the detected signal, the likelihood of a number of bases incorporated at the flow position can be determined. Solely by way of example, for the CCG sequence in Table 1, the likelihood that the flow signal indicates that 2 bases were incorporated at flow position 3 may be 0.999, and the likelihood that the flow signal indicates that 1 base was incorporated at flow position 3 may be 0.001. The sequence may be formatted as a sparse matrix, with a flow signal including a homopolymer length likelihoods for a plurality of homopolymer lengths at each flow position. Solely by way of example, a primer extended with a sequence of TATGGTCGTCGA (SEQ ID NO: 1) using a repeating flow-cycle order of T-A-C-G may result in a flowgram set shown in FIG. 2A.

Flowgrams for a respective sequence will differ based on the flow order used for sequencing. For example, Table 2 below illustrates an exemplary resulting flowgram for the three sequences CTG, CAG, and CCG. The flow order used in Table 2, solely by way of example, is A-C-T-G.

TABLE 2

| | Cycle 1 | | | | Cycle 2 | | | | Cycle 3 | | | |
|---|---|---|---|---|---|---|---|---|---|---|---|---|
| Flow Position | 1 | 2 | 3 | 4 | 5 | 6 | 7 | 8 | 9 | 10 | 11 | 12 |
| Nucleotide | A | C | T | G | A | C | T | G | A | C | T | G |
| Flow | | | | | | | | | | | | |
| CTG | 0 | 0 | 0 | 1 | 1 | 1 | 0 | 0 | 0 | 0 | 0 | 0 |
| CAG | 0 | 0 | 0 | 1 | 0 | 0 | 1 | 0 | 0 | 1 | 0 | 0 |
| CCG | 0 | 0 | 0 | 2 | 0 | 1 | 0 | 0 | 0 | 0 | 0 | 0 |

As can be seen in Table 2, for the same sequences as illustrated in Table 1, the resulting flowgram has multiple differences. In particular, three cycles rather than just two cycles of the flow order are required to fully identify the three sequences. Thus, the selection of a flow order may impact the resulting flowgram that is produced.

The homopolymer length likelihoods determined for each flow cycle may vary, for example, based on the noise or other artifacts present during detection of the analog signal during sequencing. In some embodiments, if the homopolymer length likelihood is below a predetermined threshold, the parameter may be set to a predetermined non-zero value that is substantially zero (i.e., some very small value or negligible value) to aid downstream statistical analysis further, wherein a true zero value may give rise to a computational error or insufficiently differentiate between levels of unlikelihood, e.g. very unlikely (0.0001) and inconceivable (0).

A preliminary sequence from the sequencing data set may, advantageously, be called without a sequence alignment. For example the most likely sequence, given the data, can be determined by selecting the base count with the highest likelihood at each flow position, as shown by the stars in FIG. 2B (using the same data shown in FIG. 2A). Thus, the sequence of the primer extension can be determined according to the most likely base count at each flow position: TATGGTCGTCGA (SEQ ID NO: 1). From this, the reverse complement (i.e., the template strand) can be readily determined. Further, the likelihood of this sequencing data set, given the TATGGTCGTCGA (SEQ ID NO: 1) sequence (or the reverse complement), can be determined as the product of the selected likelihood at each flow position.

At step 326 (FIG. 3B), after the preliminary sequences are called, they are mapped to a known reference sequence. The reference sequence may be a standard sequence known to a person of skill in the art. The reference may also be a sequence that has been previously determined using similar or different sequencing methods. Furthermore, the preliminary sequences may be mapped to the reference sequence in either base space or in flow space. In some embodiments where the sequences are mapped in base space, the preliminary sequence and the reference sequence may be in base space, and the mapping may be performed using approaches known to a person of skill in the art. In some embodiments where the sequences are mapped in flow space, the preliminary sequence and the reference sequence may be in flow space, and the mapping may be performed using approaches known to a person of skill in the art. Sequences in base space can be converted to flow space, as long as the flow order is known, if desired. Alternatively, sequences in flow space can be converted to base space, if desired.

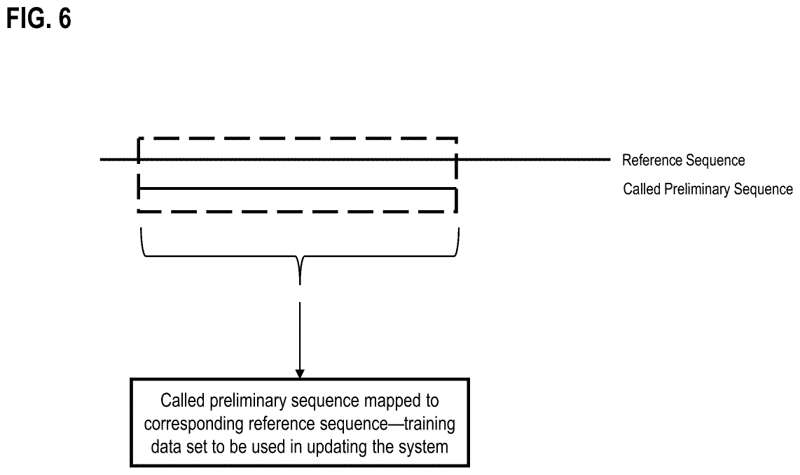

The portion of the reference sequence corresponding to the mapped preliminary sequences (i.e., the corresponding reference sequence fragments) can serve as a ground truth used to build a training data set and for further training and updating of the system, as illustrated in FIG. 6. In particular, the identified reference sequence fragment corresponding to the preliminary sequence for a given selected colony is associated with the sequencing data for that selected colony, thus generating a training data set comprising the selected sequencing data and the corresponding reference sequence fragments. The pre-trained sequencer specific machine-learning model can be updated based on the training data set.

The preliminary sequences are mapped to a known reference sequence to identify corresponding reference sequence fragments for the called preliminary sequences. Mapping the preliminary sequences to a known reference sequence establishes a ground truth for updating the system. In some embodiments, the output of the mapping step is the location in the reference genome and a fragment of the reference genome corresponding to the mapped fragment. The called preliminary sequences are outputs from the pre-trained model, but may contain sequencing errors due to inaccuracies of the pre-trained model and variances between sequencing runs. The preliminary sequences may be mapped in base space or in flow space. As described above, sequences in base space can be converted to flow space, as long as the flow order is known, if desired. Alternatively, sequences in flow space can be converted to base space, if desired.

The reference sequence may be a reference sequence from the same species. In some embodiments, the reference sequence may be from the same individual as the preliminary sequence. For example, the preliminary sequence may be isolated from a patient's cancerous tissue, while the reference sequence may be isolated from the same patient's healthy tissue. Alternatively, the reference sequence may be from a different individual than the preliminary sequence. After the preliminary sequences are mapped to the reference sequences, the ground truth data to be used in updating the system are generated.

Alignment (or mapping) of determined sequences to candidate sequences (such as candidate haplotype sequences) in base space is computationally expensive, and is currently the most computationally intensive step, for example, in the Genome Analysis Tool Kit (GATK) HaplotypeCaller. Within HaplotypeCaller, PairHMM aligns each sequencing read to each haplotype, and uses base qualities as an estimate of the error to determine the likelihood of the haplotypes given the sequencing read. However, the structure of the data set used with the methods described herein retains error mode likelihoods, which makes variant calling more computationally efficient. For example, a given genotype likelihood may be determined simply as the product of likelihoods in each flow position that aligns with the sequence having the genotype. The flow space determined likelihood can replace the PairHMM module of the HaplotypeCaller for a more computationally efficient variant call.

Thus, in step 304 (FIG. 3A), the generated training data set includes sequencing data from a selected subset of colonies, as well as the corresponding reference sequence fragments that operate as a ground truth for the training data set (e.g., as obtained from step 326). In some embodiments, the generated training data set comprises a plurality of data pairs, each data pair comprising a signal intensity vector (e.g., {a, b, c, d, . . . n} in FIG. 5) and the mapped reference sequence as the ground truth (e.g., as obtained from step 326). In some embodiments, the mapped sequence reference is expressed in homopolymer length or homopolymer length likelihoods. The training data set comprising the selected sequencing data and the corresponding reference sequence fragments can be used to update the pre-trained sequencer-specific machine-learning model. Once the pre-trained sequencing specific machine-learning model has been updated, the updated model can be used to determine the sequence for some larger portion (e.g., the entirety) of the sequencing data set.

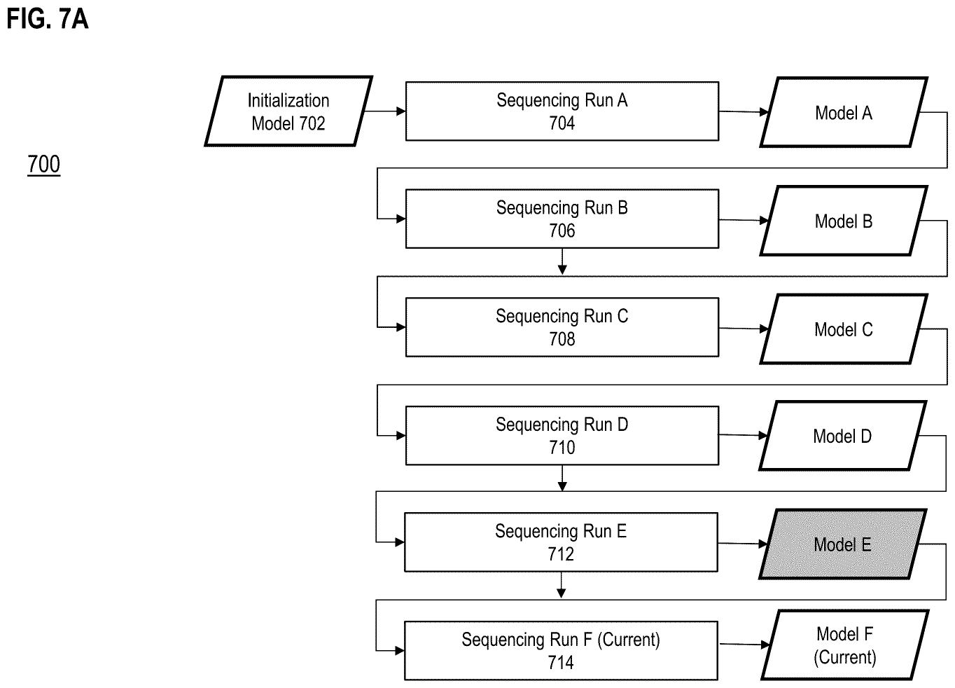

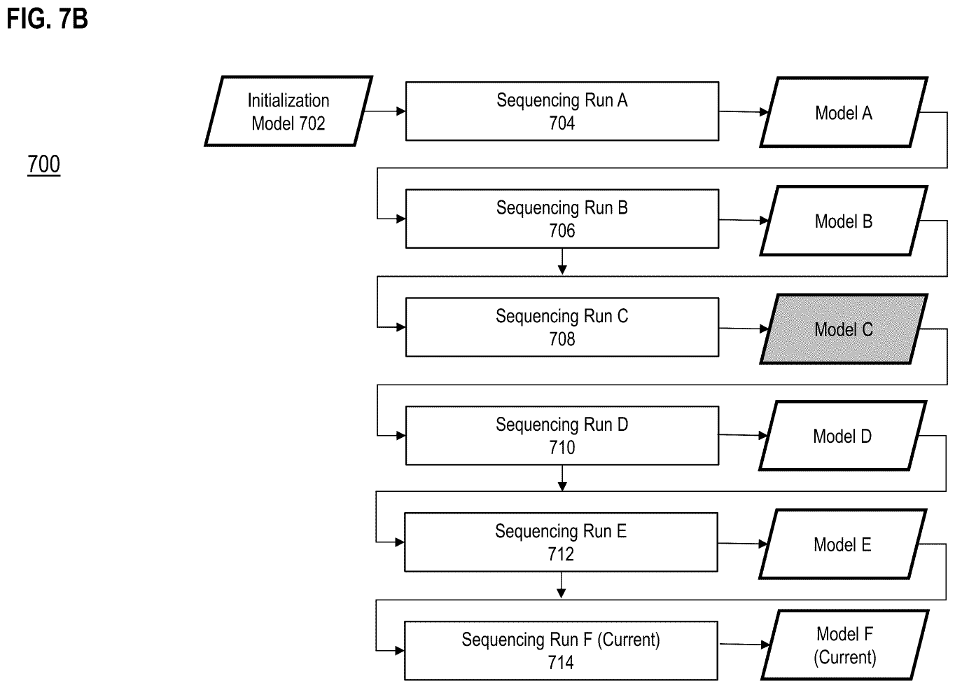

At step 306 (FIG. 3A), the pre-trained sequencer-specific machine-learning model may be a model selected from multiple models (a plurality of possible initialization models). Each of the multiple models can be trained using sequencing data generated using the same sequencer during one or more previous sequencing runs. In FIG. 7A exemplary method 700 illustrates an initialization model 702 that is used as the first model used for a given sequencer. A series of sequencing runs is performed, with Sequencing Run A 704 performed prior to Sequencing Run B 706. Sequencing Run B 706 is performed prior to Sequencing Run C 708. Sequencing Run C 708 is performed prior to Sequencing Run D 710. Sequencing Run D 710 is performed prior to Sequencing Run E 712. Sequencing Run E 712 is performed prior to the current Sequencing Run F 714. In some embodiments, any number of sequencing runs may be performed prior to the development of the current model. All sequencing runs may be performed on the same sequencer. The initialization model can be trained using data from Sequencing Run A to generate Model A. Model A can be further trained using data from Sequencing Run B to generate a Model B. Model B can be further trained using data from Sequencing Run C to generate a Model C, etc. In some embodiments, an immediately prior (i.e., penultimate) model is selected to be trained using the training data obtained in the current sequencing run. In FIG. 7A, the penultimate model for the current Sequencing Run F is Model E. Therefore, Model E can be selected to be trained based on the training data from Sequencing Run F to generate Model F. The trained Model F can then be used to process some or all of the sequencing data from Sequencing Run F (see step 310, FIG. 3A).

The Current Model may be updated as in FIG. 7A using the same sequencer and using nucleic acid molecules and sequences from the same species, which may be a primate or a human or another subject.

In some embodiments, a prior model that is not the penultimate model is selected to be trained (e.g., to be updated based on current data). In some embodiments, the pre-trained sequencer-specific machine-learning model may be a machine-learning model trained for the same sequencer on sequencing data from a prior sequencing run selected based on a quality score. With reference to FIG. 7B, rather than selecting Model E, a prior model such as Model C can be selected to be trained using training data of Sequencing Run F to generate Model F. A quality score can be associated with each of Models A-E. The quality score can be a convergence threshold, a residual error threshold, or another metric for measuring the performance of the model. In some embodiments, this quality score can be used, at least in part, to select a prior model for training. For example, a model with a corresponding quality score that is below a first threshold may be disqualified from training. Similarly, a model with a higher corresponding quality score may be selected for training over another model with a lower corresponding quality score. In FIG. 7B, solely by way of example, Model C may have an associated quality score that is higher than the associated quality scores of Models A, B, D, or E.

The Current Model may be updated as in FIG. 7B using the same sequencer and using nucleic acid molecules and sequences from the same species, which may be a primate or a human or another subject.

Regardless of the method used in updating the pre-trained sequencer-specific machine-learning model, the model may first be initialized using an initialization model. In some embodiments, the pre-trained sequencer-specific machine-learning model was previously initialized using sequencing data previously generated using the same sequencer and nucleic acid molecules from a different selected species. In some embodiments, the different selected species has a smaller genome than the selected species. In some embodiments, the different selected species is a bacterial species or a viral species. In some embodiments, the different selected species is Escherichia coli.

The pre-trained sequencer-specific machine-learning model may be, in particular, a neural network. Certain types of neural networks are commonly applied to analyze visual imagery and 2D images, which may be of beneficial use in collecting sequencing data and visual signal intensities from the sequenced nucleic acid colonies. For example, in some embodiments, the pre-trained sequencer-specific machine-learning model may be a neural network of the type that is commonly applied to analyze visual imagery and 2D images (e.g. a convoluted neural network). The machine-learning models described herein include any computer algorithms that improve automatically through experience and by the use of data. The machine-learning models can include supervised models, unsupervised models, semi-supervised models, self-supervised models, etc. Exemplary machine-learning models include but are not limited to: linear regression, logistic regression, decision tree, SVM, naive Bayes, neural networks, K-Means, random forest, dimensionality reduction algorithms, gradient boosting algorithms, etc.

At step 308 (FIG. 3A), the system can be updated using the pre-trained sequencer-specific machine-learning model based on the training data. Using this training data, the model can be iteratively trained until convergence of the model is achieved. Convergence of the adaptive model can be measured using training loss function after each epoch, when the loss function may be measured. The reduction of the loss function can be calculated relative to the loss function measured after the previous epoch, and when the reduction of the loss function reaches a threshold, which may be predetermined, the convergence step for the model can be determined. Once the difference between the loss functions between epochs falls below the previously determined threshold, the training of the software may be completed. The updated, recalibrated model can be used to call sequences for the entire data set generated in the first sequencing step of the method, as described above. The result of the final update of the system can be a recalibrated system that can be used to call the homopolymer lengths or homopolymer length likelihoods for the full sequencing data set (or some portion thereof larger than the selected subset) at step 310 (FIG. 3A).

At step 310 (FIG. 3A), the updated system can be used to call homopolymer lengths or homopolymer length likelihoods for the full dataset that was received or generated or received in step 302 (FIG. 3A) of the method. The method of determining the sequence of a target nucleotide may comprise updating the system according to any of the above described methods. To update the system, the sequencing data for the colony comprising the target nucleic acid molecule may be input into the updated sequencer-specific machine-learning model using the one or more processors.

### Systems, Devices, and Reports

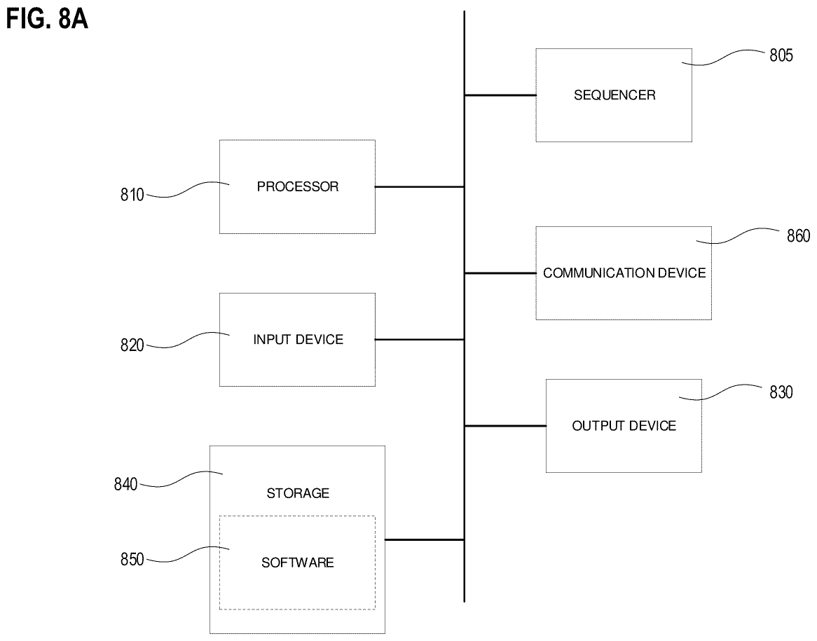

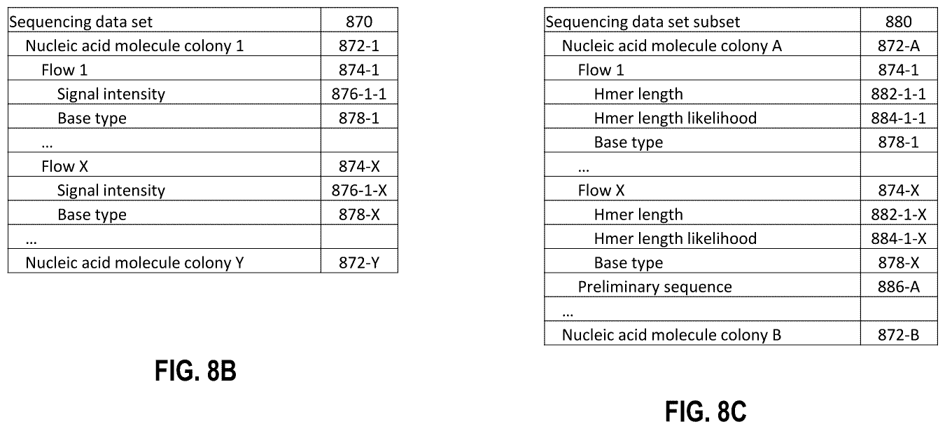

The operations described above, including those described with reference to the Figures, are optionally implemented by one or more components depicted in FIG. 8A. It would be clear to a person of ordinary skill in the art how other processes, for example, combinations or sub-combinations of all or part of the operations described above, may be implemented based on the components depicted in FIG. 8A. It would also be clear to a person having ordinary skill in the art how the methods, techniques, systems, and devices described herein may be combined with one another, in whole or in part, whether or not those methods, techniques, systems, and/or devices are implemented by and/or provided by the components depicted in FIG. 8A.

FIG. 8A illustrates an example of a computing device in accordance with some embodiments. Device 800 can be a host computer connected to a network. Device 800 can be a client computer or a server. As shown in FIG. 8, device 800 can be any suitable type of microprocessor-based device, such as a personal computer, workstation, server, or handheld computing device (portable electronic device) such as a phone or tablet. The device can include, for example, one or more of sequencer 805, processor 810, input device 820, output device 830, storage 840, and communication device 860. Input device 820 and output device 830 can generally correspond to those described above, and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage, such as an electrical, magnetic or optical memory including a RAM, cache, hard drive, or removable storage disk. Storage 840 encompasses persistent memory and non-persistent memory. Non-persistent memory includes electronically addressable solid-state memory and mechanically addressable memory (e.g., hard disks, optical disks, tape, etc.). In some embodiments, non-persistent memory includes high-speed random-access memory or other random-access solid-state memory devices. Persistent memory optionally includes one or more remote storage devices (e.g., remote from the one or more processors). In some embodiments, persistent memory and/or non-volatile memory device(s) within non-persistent memory comprises non-transitory computer readable storage medium.

Communication device 860 can include any suitable device capable of transmitting and receiving signals over a network, such as a network interface chip or device. The components of the computer can be connected in any suitable manner, such as via a physical bus or wirelessly. In some embodiments, communication device 860 includes communication buses, including circuitry that interconnects and controls communications between device 800 components.

Software 850, which can be stored in storage 840 and executed by processor 810, can include, for example, the programming that embodies the functionality of the present disclosure (e.g., as embodied in the devices as described above).

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can implement any operating system suitable for operating on the network. Software 850 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

The methods described herein optionally further include reporting information determined using the analytical methods and/or generating a report containing the information determined using the analytical methods.

As described with respect to FIG. 8A, device 800 can store, use, and process sequencing read data in accordance with methods described herein. Specifically, memory 840 (e.g., non-transitory computer readable medium) may store the following:
An operating system, including procedures for handling various basic system services and for performing hardware-dependent tasks;
A training module including instructions for training sequencer-specific machine-learning modules as described herein;
One or more pre-trained sequencer-specific machine-learning models for processing sequencing information (e.g., for determining target nucleic acid molecule sequences) as described herein;
One or more sequencing data sets, each comprising sequencing information for a plurality of nucleic acid molecule colonies;
One or more processed sequencing data sets, each comprising sequencing information for a subset of nucleic acid molecule colonies, where the subset of nucleic acid molecule colonies is selected from the plurality of nucleic acid molecule colonies, and where the subset has the same or less than the total number of nucleic acid molecule colonies in the plurality of nucleic acid molecule colonies;
An optional network communication module, or instructions, for connecting the device 1000 with other devices or a communication network;
An I/O module including procedures for handling various basic input and output functions through the input and output devices (820, 830); and
Optionally, additional modules including instructions for handling other functions and aspects described herein.

In some embodiments, one or more of the above-mentioned elements is stored in a memory as described above. The above-mentioned elements each correspond to a set of instructions for a function as described above. The above-mentioned modules, data, or programs may be implemented as separate software programs, procedure, datasets, or modules. Alternatively, or in addition, the above-mentioned modules, data, or programs may be combined or otherwise rearranged in various implementations.

Although FIG. 8A depicts device 800, this is intended as a functional description of the various features that may be present in a device rather than as a structural schematic of the implementations described herein. As will be recognized by those of skill in the art, items that are shown as combined may be separated, and some items may be combined.

In some embodiments, there is a system comprising: (a) a sequencer; (b) one or more processors; (c) computer-readable memory; (d) a pre-trained sequencer-specific machine-learning model stored in the computer-readable memory, wherein the pre-trained sequencer-specific machine-learning model is configured to call a homopolymer length or homopolymer length likelihood for each sequencing flow step based on signal intensity values, wherein the pre-trained sequencer-specific machine-learning model was previously trained based on sequencing data previously generated using the sequencer and nucleic acid molecules from a selected species; and (e) one or more programs stored in the memory and configured to be executed by the one or more processors, the one or more programs including instructions for: (i) generating, using the sequencer, sequencing data for a plurality of nucleic acid molecule colonies comprising nucleic acid molecules derived from the selected species, wherein the generating comprises extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; (ii) selecting sequencing data for a subset of the nucleic acid molecule colonies; (iii) calling preliminary sequences for the subset of the nucleic acid molecule colonies, comprising inputting the selected sequencing data into the pre-trained sequencer-specific machine-learning model; (iv) mapping the called preliminary sequences to a known reference sequence to identify corresponding reference sequence fragments for the called preliminary sequences; and (v) updating the pre-trained sequencer-specific machine-learning model based on a training data set comprising the selected sequencing data and the corresponding reference sequence fragments.

In some embodiments, the pre-trained sequencer-specific machine-learning model was previously based on penultimate sequencing data generated using the same sequencer and penultimate nucleic acid molecules from the same selected species.

In some embodiments, the pre-trained sequencer-specific machine-learning model was previously updated using a method comprising (a) generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step; (b) selecting penultimate sequencing data for a subset of the penultimate nucleic acid molecule colonies; (c) calling penultimate preliminary sequences for the subset of the penultimate nucleic acid molecule colonies, comprising inputting the selected penultimate sequencing data into a penultimate pre-trained sequencer-specific machine-learning model configured to call a homopolymer length for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species; (d) mapping the called penultimate preliminary sequences to the known reference sequence to identify corresponding penultimate reference sequence fragments for the called penultimate preliminary sequences; and (e) updating the penultimate pre-trained sequencer-specific machine-learning model based on a penultimate training data set comprising the selected penultimate sequencing data and the corresponding penultimate reference sequence fragments.

In some embodiments, the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

In some embodiments, the pre-trained sequencer-specific machine-learning model was previously initialized using sequencing data previously generated using the same sequencer and nucleic acid molecules from a different selected species. In some embodiments, the different selected species has a smaller genome than the selected species. In some embodiments, the different selected species is a bacterial species or a viral species. In some embodiments, the different selected species is Escherichia coli.

In some embodiments, the selected species is a primate. In some embodiments, the selected species is a human.

In some embodiments, the sequencer-specific machine-learning model is a neural network. In some embodiments, the sequencer-specific machine-learning model is a convoluted neural network.

In some embodiments, the sequencing data comprises, for each nucleic acid molecule colony, a vector comprising a signal intensity value at each sequencing flow step.

In some embodiments, updating the pre-trained sequencer-specific machine-learning model based on the training data set comprises iteratively updating the pre-trained sequencer-specific machine-learning model using the same training data set until a predetermined quality control threshold is met or surpassed. In some embodiments, the predetermined threshold is a convergence threshold. In some embodiments, the predetermined threshold is a residual error threshold.

In some embodiments, the selected sequencing data for the subset of the nucleic acid molecule colonies is randomly selected. In some embodiments, the selected sequencing data for the subset of the nucleic acid molecule colonies is pseudo-randomly selected. In some embodiments, the selected sequencing data for the subset of the nucleic acid molecule colonies is selected based on one or more colony parameters. In some embodiments, the one or more colony parameters include an average homopolymer length likelihood (e.g., an average of all the homopolymer length likelihoods for a nucleic acid molecule colony). In some embodiments, the one or more colony parameters include a quality metric. The quality metric may be, for example, a read quality metric or a signal (e.g., a photometry signal) quality metric.

Exemplary methods for determining a read quality metric are described in PCT/US2022/074056, the contents of which are incorporated herein by reference in its entirety and for all purposes. The read quality metric may be based on, for example, one or more homopolymer probability values other than a highest homopolymer probability value. In some embodiments, the read quality metric is a regressed residual. In some embodiments, the read quality metric for each flow step of each sequencing read is calculated based on a second highest homopolymer probability value (p_{2nd}). For example, in flow step 202 in FIG. 2A, the second highest probably value is 0.0010. In some embodiments, the read quality metric (i.e., rₛ) is calculated as:

$${r}_{s} = {log}_{10} ( {p}_{2 nd} / ϵ ) / 10 , \qquad ( 1 )$$

where ϵ is a scaling factor and p_{2nd} is the second highest probability at the flow step (e.g., representing the second most likely h-mer). In some embodiments, e can be set at a value between 1×10⁻² and 1×10⁻⁴.

The read quality metric for a given flow step can be calculated using other techniques. In some embodiments, rather than p_{2nd}, (1−p₁ₛₜ) is used in the formula above. In cases in which p₁ₛₜ+p_{2nd}=1, the two formula variations would yield the same read quality metric. In cases in which p₁ₛₜ+p_{2nd}+p_{3rd}=1, the two formula variations would yield different read quality metrics. In most cases, p_{3rd}, p₄ₜₕ, p₅ₜₕ, etc. are small numbers in comparison with p₁ₛₜ and p_{2nd}. In any such case, p₁ₛₜ+p_{2nd}+ . . . +pₙₜₕ=1.

A higher read quality metric can be indicative a weaker signal. For example, a higher p_{2nd} can indicate a lower p₁ₛₜ. Because the base count associated with p₁ₛₜ is selected a lower p₁ₛₜ can indicate a lower confidence in the selected base count. Thus, the read quality metric is used to determine flows with low confidence, which can indicate deterioration in h-mer determination accuracy, in a sequencing read and determine where (e.g., at which flow) to trim the sequencing read, as described below.

It will be understood that the read quality metric could also be calculated, with appropriate modifications to the read quality metric function, using any h-mer probability value each flow step of each sequencing read (e.g., p₁ₛₜ, p_{2nd}, p_{3rd} . . . , pₙₜₕ). Calculating the read quality metric with, for example, a first highest homopolymer probability value can be performed thus:

$${r}_{s} = {log}_{10} ( ( 1 - {p}_{1 st} ) / ϵ ) / 10 , \qquad ( 2 )$$

where ϵ would be set as in equation (1).

The signal quality metric indicates the quality of the signal (which may be, for example a photometric signal) from the colony during a sequencing run. In some embodiments, the signal quality metric may include one or more of signal amplitude, signal profile, colony location or position, colony location or positional error, average background signal, local background signal, maximum gray-level, number of saturated pixels, a measure of the goodness of fit of the signal profile relative to a known profile (for example, based on a full width at half maximum (“FWHM”) estimate, a pseudo-Voigt Lorentzian weight (tail parameter), or one or more parameters of an elliptic model used to fit the signal), and/or signal-to-noise ratio

In some embodiments, the plurality of nucleic acid molecule colonies comprise a colony comprising the target nucleic acid molecule, and the one or more programs further include instructions for: (a) inputting the sequencing data for the colony comprising the target nucleic acid molecule into the updated sequencer-specific machine-learning model; and (b) calling, for the target nucleic acid molecule, a homopolymer length for each sequencing flow step using the updated sequencer-specific machine-learning model.

In some embodiments, the methods described herein are computer-implemented methods, which may be performed using one or more of the components illustrated in FIG. 7. For example, in some embodiments, a computer-readable memory comprises: (a) a pre-trained sequencer-specific machine-learning model configured to call a homopolymer length or homopolymer length likelihood for each sequencing flow step based on signal intensity values, wherein the pre-trained sequencer-specific machine-learning model was previously trained based on sequencing data previously generated using a sequencer and nucleic acid molecules from a selected species; and (b) one or more programs comprising instructions, which executed by one or more processors of an electronic device, cause the electronic device to: (i) receive sequencing data for a plurality of nucleic acid molecule colonies comprising nucleic acid molecules derived from the selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step; (ii) select sequencing data for a subset of the nucleic acid molecule colonies; (iii) call preliminary sequences for the subset of the nucleic acid molecule colonies, comprising inputting the selected sequencing data into the pre-trained sequencer-specific machine-learning model; (iv) map the called preliminary sequences to a known reference sequence to identify corresponding reference sequence fragments for the called preliminary sequences; and (v) update the pre-trained sequencer-specific machine-learning model based on a training data set comprising the selected sequencing data and the corresponding reference sequence fragments.

In some embodiments, the pre-trained sequencer-specific machine-learning model was previously updated based on penultimate sequencing data generated using the same sequencer and penultimate nucleic acid molecules from the same selected species.

In some embodiments, the pre-trained sequencer-specific machine-learning model was previously updated by a method comprising: (a) generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step; (b) selecting penultimate sequencing data for a subset of the penultimate nucleic acid molecule colonies; (c) calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species; (d) mapping the called penultimate preliminary sequences to the known reference sequence to identify corresponding penultimate reference sequence fragments for the called penultimate preliminary sequences; and (e) updating the penultimate pre-trained sequencer-specific machine-learning model based on a penultimate training data set comprising the selected penultimate sequencing data and the corresponding penultimate reference sequence fragments.

In some embodiments, the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

In some embodiments, the pre-trained sequencer-specific machine-learning model was previously initialized using sequencing data previously generated using the same sequencer and nucleic acid molecules from a different selected species.

In some embodiments, the different selected species has a smaller genome than the selected species. In some embodiments, the different selected species is a bacterial species or a viral species. In some embodiments, the different selected species is Escherichia coli.

In some embodiments, the selected species is a primate. In some embodiments, the selected species is a human.

In some embodiments, the sequencer-specific machine-learning model is a neural network. In some embodiments, the sequencer-specific machine-learning model is a convoluted neural network.

In some embodiments, the sequencing data comprises, for each nucleic acid colony, a vector comprising a signal intensity value at each sequencing flow step.

In some embodiments, updating the pre-trained sequencer-specific machine-learning model based on the training data set comprises iteratively updating the pre-trained sequencer-specific machine-learning model using the same training data set until a predetermined quality control threshold is met or surpassed. In some embodiments, the quality control threshold is a convergence threshold. In some embodiments, the quality control threshold is a residual error threshold.

In some embodiments, updating the pre-trained sequencer-specific machine-learning model based on the training data set comprises iteratively updating the pre-trained sequencer-specific machine-learning model using the same training data set until a change in a quality control value between iterations is below a predetermined threshold. In some embodiments, the predetermined threshold is a convergence threshold. In some embodiments, the predetermined threshold is a residual error threshold.

### Exemplary Data Structures

While methods in accordance with the present disclosure have been discussed above, more details as to the type of data that may be processed or provided by these methods are now described. FIGS. 8B and 8C illustrate example block diagrams of sequencing data sets in accordance with embodiments described herein.

FIG. 8B shows an example of a sequencing data set. Sequencing data set 870 comprises data for a first plurality of nucleic acid molecule colonies 872, where information for each nucleic acid molecule colony comprises, for each flow in a plurality of sequencing flow steps, a signal intensity value 876 and a base type. The base type for each sequencing flow is determined by the sequencing method (e.g., nucleic acid base types are added discretely in series in order to extend sequencing primers, as described elsewhere herein). In some embodiments, sequencing data set 870 as depicted in FIG. 8B may comprise sequencing information for a single individual of a species (or for a single experiment). In some embodiments, sequencing data set 870 as depicted in FIG. 8B may comprise sequencing information for multiple individuals or one or more multiple species (or for multiple experiments). In either case, a sequencing data set 870 will include sequencing information obtained from a single sequencing machine (e.g., a same sequencer). In some embodiments, there will be multiple sequencing data sets 870, where one or more were obtained from a first sequencer and another one or more were obtained from a second sequencer.

FIG. 8C shows an example of a selected sequencing data set (e.g., a subset of a sequencing data set 870). Sequencing data set subset 880 comprises data for a second plurality of nucleic acid molecule colonies 872, where the second plurality of nucleic acid molecule colonies 872 is a subset of the first plurality of nucleic acid molecule colonies. Data for each nucleic acid molecule colony 872 in the second plurality of nucleic acid molecule colonies comprises, for each flow in the plurality of sequencing flow steps, i) a homopolymer length (hmer length 882) or a homopolymer length likelihood (hmer length likelihood 884) and ii) the base type of the respective flow. In addition, data for each nucleic acid molecule colony in the second plurality of nucleic acid molecule colonies comprises a respective preliminary sequence, where the preliminary sequences are determined from the pre-trained sequencer-specific machine-learning model that is used to process the selected sequencing data set (e.g., the pre-trained sequencer-specific machine-learning model that is updated or retrained using the selected sequencing data set).

In such embodiments, subsets of sequencing data sets obtained from the first sequencer may be used to train (e.g., retrain or update) a first pre-trained sequencer-specific machine-learning model that has been pre-trained using additional sequencing data sets, e.g., penultimate sequencing data sets, or subsets thereof, obtained from the first sequencer (e.g., the first pre-trained sequencer-specific machine-learning model is specific to the first sequencer).

### Exemplary Embodiments

Among the provided embodiments are:
Embodiment 1. A method of updating a system comprising a sequencer, the method comprising:
receiving, at one or more processors, sequencing data for a plurality of nucleic acid molecule colonies comprising nucleic acid molecules derived from a selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step;
selecting, using the one or more processors, sequencing data for a subset of the nucleic acid molecule colonies;
calling, using the one or more processors, preliminary sequences for the subset of the nucleic acid molecule colonies, comprising inputting the selected sequencing data into a pre-trained sequencer-specific machine-learning model configured to call a homopolymer length or a homopolymer length likelihood for each sequencing flow step based on the signal intensity values, wherein the pre-trained sequencer-specific machine-learning model was previously trained based on sequencing data previously generated using the same sequencer and nucleic acid molecules from the same selected species;
mapping, using the one or more processors, the called preliminary sequences to a known reference sequence to identify corresponding reference sequence fragments for the called preliminary sequences; and
updating, using the one or more processors, the pre-trained sequencer-specific machine-learning model based on a training data set comprising the selected sequencing data and the corresponding reference sequence fragments.
Embodiment 2. The method of embodiment 1, comprising generating, using the sequencer, the sequencing data.
Embodiment 3. The method of embodiment 1, wherein the pre-trained sequencer-specific machine-learning model was previously updated based on penultimate sequencing data generated using the same sequencer and penultimate nucleic acid molecules from the same selected species.
Embodiment 4. The method of embodiment 3, wherein the pre-trained sequencer-specific machine-learning model was previously updated by a method comprising:
generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step;
selecting penultimate sequencing data for a subset of the penultimate nucleic acid molecule colonies;
calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species;
mapping the called penultimate preliminary sequences to the known reference sequence to identify corresponding penultimate reference sequence fragments for the called penultimate preliminary sequences; and
updating the penultimate pre-trained sequencer-specific machine-learning model based on a penultimate training data set comprising the selected penultimate sequencing data and the corresponding penultimate reference sequence fragments.
Embodiment 5. The method of embodiment 1, wherein the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.
Embodiment 6. The method of any one of embodiments 1-5, wherein the pre-trained sequencer-specific machine-learning model was previously initialized using sequencing data previously generated using the same sequencer and nucleic acid molecules from a different selected species.
Embodiment 7. The method of embodiment 6, wherein the different selected species has a smaller genome than the selected species.
Embodiment 8. The method of embodiment 6 or 7, wherein the different selected species is a bacterial species or a viral species.
Embodiment 9. The method of any one of embodiments 6-8, wherein the different selected species is Escherichia coli.
Embodiment 10. The method of any one of embodiments 1-9, wherein the selected species is a primate.
Embodiment 11. The method of any one of embodiments 1-10, wherein the selected species is a human.
Embodiment 12. The method of any one of embodiments 1-11, wherein the sequencer-specific machine-learning model is a neural network.
Embodiment 13. The method of any one of embodiments 1-12, wherein the sequencer-specific machine-learning model is a convoluted neural network.
Embodiment 14. The method of any one of embodiments 1-13, wherein the sequencing data comprises, for each nucleic acid molecule colony, a vector comprising a signal intensity value at each sequencing flow step.
Embodiment 15. The method of any one of embodiments 1-14, wherein updating the pre-trained sequencer-specific machine-learning model based on the training data set comprises iteratively updating the pre-trained sequencer-specific machine-learning model using the same training data set until a predetermined quality control threshold is met or surpassed.
Embodiment 16. The method of embodiment 15, wherein the predetermined quality control threshold is a convergence threshold.
Embodiment 17. The method of any one of embodiments 1-14, wherein updating the pre-trained sequencer-specific machine-learning model based on the training data set comprises iteratively updating the pre-trained sequencer-specific machine-learning model using the same training data set until a change in a quality control value between iterations is below a predetermined threshold.
Embodiment 18. The method of embodiment 15, wherein the predetermined threshold is a convergence threshold.
Embodiment 19. The method of any one of embodiments 1-18, wherein the selected sequencing data for the subset of the nucleic acid molecule colonies is randomly selected.
Embodiment 20. A method of determining a sequence of a target nucleic acid molecule, comprising:
updating a system according to the method of any one of embodiments 1-19, wherein the plurality of nucleic acid molecule colonies comprises a colony comprising the target nucleic acid molecule;
inputting, using the one or more processors, the sequencing data for the colony comprising the target nucleic acid molecule into the updated sequencer-specific machine-learning model; and
calling, using the one or more processors, for the target nucleic acid molecule, a homopolymer length for each sequencing flow step using the updated sequencer-specific machine-learning model.
Embodiment 21. A system, comprising
a sequencer;
one or more processors;
a computer-readable memory;
a pre-trained sequencer-specific machine-learning model stored in the computer-readable memory, wherein the pre-trained sequencer-specific machine-learning model is configured to call a homopolymer length or a homopolymer length likelihood for each sequencing flow step based on signal intensity values, wherein the pre-trained sequencer-specific machine-learning model was previously trained based on sequencing data previously generated using the sequencer and nucleic acid molecules from a selected species; and
one or more programs stored in the memory and configured to be executed by the one or more processors, the one or more programs including instructions for:
receiving at the one or more processors, from the sequencer, sequencing data for a plurality of nucleic acid molecule colonies comprising nucleic acid molecules derived from the selected species, wherein the sequencing data is generated using a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step;
selecting, using the one or more processors, sequencing data for a subset of the nucleic acid molecule colonies;
calling, using the one or more processors, preliminary sequences for the subset of the nucleic acid molecule colonies, comprising inputting the selected sequencing data into the pre-trained sequencer-specific machine-learning model;
mapping, using the one or more processors, the called preliminary sequences to a known reference sequence to identify corresponding reference sequence fragments for the called preliminary sequences; and
updating, using the one or more processors, the pre-trained sequencer-specific machine-learning model based on a training data set comprising the selected sequencing data and the corresponding reference sequence fragments.
Embodiment 22. The system of embodiment 21, wherein the pre-trained sequencer-specific machine-learning model was previously updated based on penultimate sequencing data generated using the same sequencer and penultimate nucleic acid molecules from the same selected species.
Embodiment 23. The system of embodiment 22, wherein the pre-trained sequencer-specific machine-learning model was previously updated by a method comprising:
the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step;
selecting penultimate sequencing data for a subset of the penultimate nucleic acid molecule colonies;
calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species;
mapping the called penultimate preliminary sequences to the known reference sequence to identify corresponding penultimate reference sequence fragments for the called penultimate preliminary sequences; and
updating the penultimate pre-trained sequencer-specific machine-learning model based on a penultimate training data set comprising the selected penultimate sequencing data and the corresponding penultimate reference sequence fragments.
Embodiment 24. The system of embodiment 21, wherein the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.
Embodiment 25. The system of any one of embodiments 21-24, wherein the pre-trained sequencer-specific machine-learning model was previously initialized using sequencing data previously generated using the same sequencer and nucleic acid molecules from a different selected species.
Embodiment 26. The system of embodiment 25, wherein the different selected species has a smaller genome than the selected species.
Embodiment 27. The system of embodiment 25 or 26, wherein the different selected species is a bacterial species or a viral species.
Embodiment 28. The system of any one of embodiments 25-27, wherein the different selected species is Escherichia coli.
Embodiment 29. The system of any one of embodiments 21-28, wherein the selected species is a primate.
Embodiment 30. The system of any one of embodiments 21-29, wherein the selected species is a human.
Embodiment 31. The system of any one of embodiments 21-30, wherein the sequencer-specific machine-learning model is a neural network.
Embodiment 32. The system of any one of embodiments 21-31, wherein the sequencer-specific machine-learning model is a convoluted neural network.
Embodiment 33. The system of any one of embodiments 21-32, wherein the sequencing data comprises, for each nucleic acid molecule colony, a vector comprising a signal intensity value at each sequencing flow step.
Embodiment 34. The system of any one of embodiments 21-33, wherein updating the pre-trained sequencer-specific machine-learning model based on the training data set comprises iteratively updating the pre-trained sequencer-specific machine-learning model using the same training data set until a predetermined quality control threshold is met or surpassed.
Embodiment 35. The system of embodiment 34, wherein the predetermined quality control threshold is a convergence threshold.
Embodiment 36. The system of any one of embodiments 21-35, wherein updating the pre-trained sequencer-specific machine-learning model based on the training data set comprises iteratively updating the pre-trained sequencer-specific machine-learning model using the same training data set until a change in a quality control value between iterations is below a predetermined threshold.
Embodiment 37. The system of embodiment 36, wherein the predetermined threshold is a convergence threshold.
Embodiment 38. The system of any one of embodiments 21-37, wherein the selected sequencing data for the subset of the nucleic acid molecule colonies is randomly selected.
Embodiment 39. The system of any one of embodiments 21-38, wherein the plurality of nucleic acid molecule colonies comprises a colony comprising the target nucleic acid molecule, and wherein the one or more programs further include instructions for;
inputting the sequencing data for the colony comprising the target nucleic acid molecule into the updated sequencer-specific machine-learning model; and
calling, for the target nucleic acid molecule, a homopolymer length for each sequencing flow step using the updated sequencer-specific machine-learning model.
Embodiment 40. A computer-readable memory storing:
a pre-trained sequencer-specific machine-learning model configured to call a homopolymer length or a homopolymer length likelihood for each sequencing flow step based on signal intensity values, wherein the pre-trained sequencer-specific machine-learning model was previously trained based on sequencing data previously generated using a sequencer and nucleic acid molecules from a selected species; and
one or more programs comprising instructions, which executed by one or more processors of an electronic device, cause the electronic device to:
receive sequencing data for a plurality of nucleic acid molecule colonies comprising nucleic acid molecules derived from the selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step;
select sequencing data for a subset of the nucleic acid molecule colonies;
call preliminary sequences for the subset of the nucleic acid molecule colonies, comprising inputting the selected sequencing data into the pre-trained sequencer-specific machine-learning model;
map the called preliminary sequences to a known reference sequence to identify corresponding reference sequence fragments for the called preliminary sequences; and
update the pre-trained sequencer-specific machine-learning model based on a training data set comprising the selected sequencing data and the corresponding reference sequence fragments.
Embodiment 41. The computer-readable memory of embodiment 40, wherein the pre-trained sequencer-specific machine-learning model was previously updated based on penultimate sequencing data generated using the same sequencer and penultimate nucleic acid molecules from the same selected species.
Embodiment 42. The computer-readable memory of embodiment 41, wherein the pre-trained sequencer-specific machine-learning model was previously updated by a method comprising:
generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step;
selecting penultimate sequencing data for a subset of the penultimate nucleic acid molecule colonies;
calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species;
mapping the called penultimate preliminary sequences to the known reference sequence to identify corresponding penultimate reference sequence fragments for the called penultimate preliminary sequences; and
updating the penultimate pre-trained sequencer-specific machine-learning model based on a penultimate training data set comprising the selected penultimate sequencing data and the corresponding penultimate reference sequence fragments.
Embodiment 43. The computer-readable memory of embodiment 40, wherein the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.
Embodiment 44. The computer-readable memory of any one of embodiments 40-43, wherein the pre-trained sequencer-specific machine-learning model was previously initialized using sequencing data previously generated using the same sequencer and nucleic acid molecules from a different selected species.
Embodiment 45. The computer-readable memory of embodiment 44, wherein the different selected species has a smaller genome than the selected species.
Embodiment 46. The computer-readable memory of embodiment 44 or 45, wherein the different selected species is a bacterial species or a viral species.
Embodiment 47. The computer-readable memory of any one of embodiments 44-46, wherein the different selected species is Escherichia coli.
Embodiment 48. The computer-readable memory of any one of embodiments 40-47, wherein the selected species is a primate.
Embodiment 49. The computer-readable memory of any one of embodiments 40-48, wherein the selected species is a human.
Embodiment 50. The computer-readable memory of any one of embodiments 40-49, wherein the sequencer-specific machine-learning model is a neural network.
Embodiment 51. The computer-readable memory of any one of embodiments 40-50, wherein the sequencer-specific machine-learning model is a convoluted neural network.
Embodiment 52. The computer-readable memory of any one of embodiments 40-51, wherein the sequencing data comprises, for each nucleic acid molecule colony, a vector comprising a signal intensity value at each sequencing flow step.
Embodiment 53. The computer-readable memory of any one of embodiments 40-52, wherein updating the pre-trained sequencer-specific machine-learning model based on the training data set comprises iteratively updating the pre-trained sequencer-specific machine-learning model using the same training data set until a predetermined quality control threshold is met or surpassed.
Embodiment 54. The computer-readable memory of embodiment 53, wherein the predetermined quality control threshold is a convergence threshold.
Embodiment 55. The computer-readable memory of any one of embodiments 40-54, wherein updating the pre-trained sequencer-specific machine-learning model based on the training data set comprises iteratively updating the pre-trained sequencer-specific machine-learning model using the same training data set until a change in a quality control value between iterations is below a predetermined threshold.
Embodiment 56. The computer-readable memory of embodiment 55, wherein the predetermined threshold is a convergence threshold.

### EXAMPLES

The application may be better understood by reference to the following non-limiting example, which is provided as an exemplary embodiment of the application. The following example is presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1—Convergence of Adaptive Modeling for Base-Calling Algorithms

Sequencing data for a plurality of nucleic acid molecule colonies was generated as illustrated in FIG. 1. Sequencing primers hybridized to the nucleic acid molecules were extended using a plurality of flow steps. In each flow step, a base (a mix of labeled and unlabeled dNTP) was added. The nucleic acid molecule colonies were then imaged through the measurement of a signal intensity value indicating nucleotide incorporation. After the colonies were imaged and a sum signal from each colony was determined, the label was removed. This process was repeated four total times until each of dATP, dCTP, dGTP, and dTTP were individually added, the colonies imaged, and the label on any labeled nucleotides removed.

Base calling was performed on individual sequencing wafers using a trained neural network. A first model was trained using randomized weights, and a second, adaptive-model was trained using predetermined weights. The predetermined weights were established from a preexisting neural network that was used as a starting point for training the second, adaptive model.

Loss of function was measured for the first and the second models to determine the number of training steps, or epochs, required to achieve model convergence. Loss of function is a general measure for training accuracy that can be run on a validation sample of the data after each epoch. To determine the convergence step for a model, reduction of loss function was monitored and measured until it fell below a predetermined threshold.

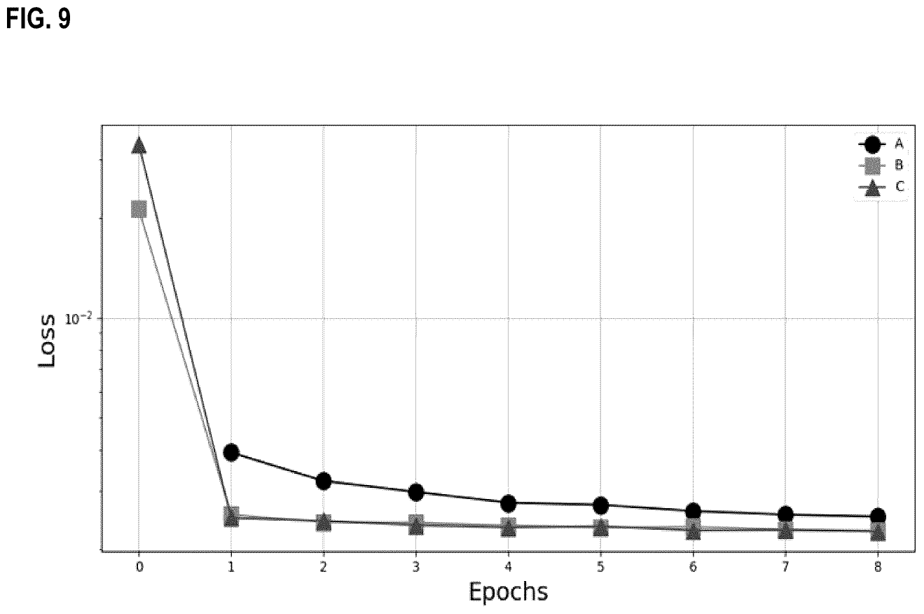

The results are illustrated in FIG. 9, which shows that the model trained on randomized weights achieves model convergence after eight epochs (e.g., the first model, A), while training the same data set on one of two preexisting models (e.g., trained from previous run B, or trained from a previous run, C, where run B and run C varied in initial parameters and/or training data), achieves convergence after only two epochs. This illustrates the advantage of training an adaptive model using predetermined weights (e.g., from another, pre-existing model). Furthermore, use of a pre-existing neural network that is retrained de novo for each sequencing run can take up to six hours, while starting from a pre-trained neural network reduces the training time required for achieving model convergence by approximately four hours. Under these conditions, adaptive training results in a four-fold reduction in the number of epochs required to train the neural network used in the base-calling algorithm and can save up to approximately four hours while analyzing read data, thereby increasing analysis throughput and alleviating sequencing data backlog.

1. A method of updating a system comprising a sequencer, the method comprising:
receiving, at one or more processors, sequencing data for a plurality of nucleic acid molecule colonies comprising nucleic acid molecules derived from a selected species, wherein the sequencing data was generated according to a method comprising extending sequencing primers hybridized to the nucleic acid molecules using a plurality of sequencing flow steps, each sequencing flow step comprising combining the plurality of nucleic acid molecule colonies with nucleotides, wherein at least a portion of the nucleotides are labeled, and measuring, for each nucleic acid molecule colony, a signal intensity value indicating nucleotide incorporation into the sequencing primers hybridized to said nucleic acid molecules, wherein the sequencing data comprises, for each nucleic acid molecule colony, a signal intensity value at each sequencing flow step;
selecting, using the one or more processors, sequencing data for a subset of the nucleic acid molecule colonies;
calling, using the one or more processors, preliminary sequences for the subset of the nucleic acid molecule colonies, comprising inputting the selected sequencing data into a pre-trained sequencer-specific machine-learning model configured to call a homopolymer length or a homopolymer length likelihood for each sequencing flow step based on the signal intensity values, wherein the pre-trained sequencer-specific machine-learning model was previously trained based on sequencing data previously generated using the same sequencer and nucleic acid molecules from the same selected species;
mapping, using the one or more processors, the called preliminary sequences to a known reference sequence to identify corresponding reference sequence fragments for the called preliminary sequences; and
updating, using the one or more processors, the pre-trained sequencer-specific machine-learning model based on a training data set comprising the selected sequencing data and the corresponding reference sequence fragments.

2. The method of claim 1, comprising generating, using the sequencer, the sequencing data.

3. The method of claim 1, wherein the pre-trained sequencer-specific machine-learning model was previously updated based on penultimate sequencing data generated using the same sequencer and penultimate nucleic acid molecules from the same selected species.

4. The method of claim 3, wherein the pre-trained sequencer-specific machine-learning model was previously updated by a method comprising:
generating the penultimate sequencing data for a plurality of penultimate nucleic acid molecule colonies comprising the penultimate nucleic acid molecules, comprising extending sequencing primers hybridized to the nucleic acid molecules using a plurality of sequencing flow steps, each sequencing flow step comprising combining the plurality of penultimate nucleic acid molecule colonies with nucleotides, wherein at least a portion of the nucleotides are labeled, and measuring, for each penultimate nucleic acid molecule colony, a signal intensity value indicating nucleotide incorporation into the sequencing primers hybridized to said nucleic acid molecules, wherein the penultimate sequencing data comprises, for each penultimate nucleic acid molecule colony, a signal intensity value at each sequencing flow step;
selecting penultimate sequencing data for a subset of the penultimate nucleic acid molecule colonies;
calling penultimate preliminary sequences for the subset of the penultimate nucleic acid molecule colonies, comprising inputting the selected penultimate sequencing data into a penultimate pre-trained sequencer-specific machine-learning model configured to call a homopolymer length or a homopolymer length likelihood for each sequencing flow step based on the signal intensity values, wherein the penultimate pre-trained sequencer-specific machine-learning model was previously trained based on antepenultimate sequencing data previously generated using the same sequencer and antepenultimate nucleic acid molecules from the same selected species;
mapping the called penultimate preliminary sequences to the known reference sequence to identify corresponding penultimate reference sequence fragments for the called penultimate preliminary sequences; and
updating the penultimate pre-trained sequencer-specific machine-learning model based on a penultimate training data set comprising the selected penultimate sequencing data and the corresponding penultimate reference sequence fragments.

5. The method of claim 1, wherein the pre-trained sequencer-specific machine-learning model is selected from a plurality of pre-trained sequencer-specific machine-learning models based on a quality score, wherein the plurality of pre-training sequencer-specific machine-learning models were each trained based on sequencing data generated using the same sequencer and nucleic acid molecules from the same selected species.

6. The method of claim 1, wherein the pre-trained sequencer-specific machine-learning model was previously initialized using sequencing data previously generated using the same sequencer and nucleic acid molecules from a different selected species.

7. The method of claim 6, wherein the different selected species has a smaller genome than the selected species.

8. The method of claim 6, wherein the different selected species is a bacterial species or a viral species.

9. The method of claim 6, wherein the different selected species is Escherichia coli.

10. The method of claim 1, wherein the selected species is a primate.

11. The method of claim 1, wherein the selected species is a human.

12. The method of claim 1, wherein the sequencer-specific machine-learning model is a neural network.

13. The method of claim 1, wherein the sequencer-specific machine-learning model is a convoluted neural network.

14. The method of claim 1, wherein the sequencing data comprises, for each nucleic acid molecule colony, a vector comprising a signal intensity value at each sequencing flow step.

15. The method of claim 1, wherein updating the pre-trained sequencer-specific machine-learning model based on the training data set comprises iteratively updating the pre-trained sequencer-specific machine-learning model using the same training data set until a predetermined quality control threshold is met or surpassed.

16. The method of claim 15, wherein the predetermined quality control threshold is a convergence threshold.

17. The method of claim 1, wherein updating the pre-trained sequencer-specific machine-learning model based on the training data set comprises iteratively updating the pre-trained sequencer-specific machine-learning model using the same training data set until a change in a quality control value between iterations is below a predetermined threshold.

18. The method of claim 15, wherein the predetermined threshold is a convergence threshold.

19. The method of claim 1, wherein the selected sequencing data for the subset of the nucleic acid molecule colonies is randomly selected.

20. A method of determining a sequence of a target nucleic acid molecule, comprising:
updating a system according to the method of claim 1, wherein the plurality of nucleic acid molecule colonies comprises a colony comprising the target nucleic acid molecule;
inputting, using the one or more processors, the sequencing data for the colony comprising the target nucleic acid molecule into the updated sequencer-specific machine-learning model; and
calling, using the one or more processors, for the target nucleic acid molecule, a homopolymer length for each sequencing flow step using the updated sequencer-specific machine-learning model.

21-56. (canceled)
